(19) United States
(12) Patent Application Publication
(10) Pub. No.: US 2024/0260855 A1
(43) Pub. Date: Aug. 8, 2024

(54) CONDITION SETTING DEVICE, MEASUREMENT DEVICE, DATA PROCESSING DEVICE, CONDITION SETTING METHOD, AND RECORDING MEDIUM

(71) Applicant: NEC Corporation, Minato-ku, Tokyo (JP)

(72) Inventor: Hiroshi KAJITANI, Tokyo (JP)

(73) Assignee: NEC Corporation, Minato-ku, Tokyo (JP)

(21) Appl. No.: 18/565,176

(22) PCT Filed: Jun. 4, 2021
(86) PCT No.: PCT/JP2021/021320

§ 371 Date: Nov. 29, 2023

(51) Int. Cl. A61B 5/11; A61B 5/00

(52) U.S. Cl. CPC A61B 5/112; A61B 5/6829; A61B 5/7475; A61B 2560/0209; A61B 2560/0219; A61B 2562/0219

(57) ABSTRACT

A condition setting device includes an acquisition unit that acquires measurement success/failure information indicating success/failure of measurement of a physical quantity related to a motion of a foot, the measurement being executed by a measurement device installed at a foot portion of a user at a measurement opportunity included in a target measurement time zone, and a measurement condition setting unit that sets, based on the measurement success/failure information in the target measurement time zone, a measurement condition including a standby time from when a gait is sensed at a measurement opportunity included in a related measurement time zone related to the target measurement time zone to when measurement is started.

### TECHNICAL FIELD

The present disclosure relates to a condition setting device or the like that sets a measurement condition in gait measurement.

### BACKGROUND ART

With increasing interest in healthcare that performs physical condition management, a service that measures features (also referred to as a gait) included in a gait pattern and provides information related to the gait to a user has attracted attention. For example, a device has been developed in which a pressure measurement device or an inertial measurement device is mounted on footwear such as shoes and the gait of a user is analyzed. In the gait measurement using such a measurement device, since the battery capacity is limited, the measurement time zone is limited in order to reduce the power consumption. The measurement time zone includes several measurement opportunities, but when the measurement fails in all the measurement opportunities, the measurement in the measurement time zone fails. Therefore, in order to reduce the power consumption of the measurement device, it is required to efficiently set the timing of measurement by the measurement device.

PTL 1 discloses a sensor control device that controls the operation of a pressure sensor inserted into a shoe. The device of PTL 1 detects the start and end of the swing period based on sensor data output by a motion sensor. The device of PTL 1 stops the operation of the pressure sensor at the timing when the start of the swing period is sensed, and starts the operation of the pressure sensor at the timing when the end of the swing period is sensed, thereby suppressing power consumption in the swing period.

PTL 2 discloses an energization control device for a water heater for cleaning water and a heating toilet seat. The device of PTL 2 predicts an unused time zone of a user based on outputs of a clocking means and a human body detection means. The device of PTL 2 performs energization control to de-energize the device for the predicted unused time zone. The device of PTL 2 sets a use frequency level for each of time zones obtained by dividing one day by a predetermined time unit. In the device of PTL 2, the use frequency levels of a plurality of time zones before and after the time zone in which the output of the human body detection means is present are set to be large, the use frequency level of the time zone in which the output of the human body detection means is not present is gradually reduced, and the time zone in which the use frequency level is minimum is set as the unused time zone.

PTL 3 discloses a sanitary washing device that controls power supply to a circuit unit to be power saved such as a water heater or a toilet seat heater. The device of PTL 3 stores use history information indicating a use situation of the toilet in each time zone provided by dividing one day into a plurality of time zones, and sequentially stores use history information indicating that the toilet is used in the corresponding time zone according to the use of the toilet. The device of PTL 3 constantly supplies power to the circuit unit to be power saved during the normal operation. In a case where switching to the automatic power saving operation is performed according to a predetermined operation, the device of PTL 3 determines whether the power saving operation in each time zone is necessary based on the stored use history information of the same time zone. In the case of the time zone in which power saving is unnecessary, the device of PTL 3 supplies power to the circuit unit to be power saved. In the case of the time zone requiring power saving, the device of PTL 3 automatically performs the power saving operation by stopping the power supply to the circuit unit to be power saved.

### CITATION LIST

### Patent Literature

PTL 1: JP 2020-058688 A
PTL 2: JP 2000-303536 A
PTL 3: JP 11-93243 A

### SUMMARY OF INVENTION

### Technical Problem

In the method of PTL 1, the operation of the pressure sensor is stopped during the swing period in which the measurement by the pressure sensor is not performed, thereby reducing the power consumption of the battery. In the method of PTL 1, since sensor data cannot be measured during the swing period, the gait during the swing period cannot be measured.

In the method of PTL 2, the unused time zone of the user is predicted based on the outputs of the clocking means and the human body detection means so that the power consumption by the water heater for cleaning water and the heating toilet seat is reduced. In the method of PTL 2, the output by the human body detection means is used when predicting the unused time zone. The pyroelectric infrared sensor as an example of the human body detection means generates standby power although its power consumption is smaller than that of a water heater for cleaning water or a heated toilet seat. Therefore, the method of PTL 2 is easily applied to an environment where power can be supplied at all times as in a toilet, but is difficult to apply to an environment where it is difficult to continuously supply power at all times as in the time of the gait.

In the method of PTL 3, power saving is performed by determining power saving necessity based on use history information in a time zone in which power saving is necessary. In the method of PTL 3, the use of the toilet is sensed according to the detection of the seating by the seating sensing unit. Therefore, in the method of PTL 3, there is a case where the temperature of the water heater or the toilet seat heater is not adjusted to an appropriate temperature when seating is sensed in a time zone requiring power saving. In the method of PTL 3, for the toilet used by a plurality of people, it is not possible to efficiently reduce the time zone in which power saving is unnecessary, and it is not always possible to efficiently save power.

An object of the present disclosure is to provide a condition setting device and the like capable of setting a measurement condition for performing gait measurement at an appropriate timing while reducing power consumption.

### Solution to Problem

A condition setting device according to an aspect of the present disclosure includes an acquisition unit that acquires measurement success/failure information indicating success/failure of measurement of a physical quantity related to a motion of a foot, the measurement being executed by a measurement device installed at a foot portion of a user at a measurement opportunity included in a target measurement time zone, and a measurement condition setting unit that sets, based on the measurement success/failure information in the target measurement time zone, a measurement condition including a standby time from when a gait is sensed at a measurement opportunity included in a related measurement time zone related to the target measurement time zone to when measurement is started.

In a condition setting method executed by a computer according to an aspect of the present disclosure, the method includes acquiring measurement success/failure information indicating success/failure of measurement of a physical quantity related to a motion of a foot, the measurement being executed by a measurement device installed at a foot portion of a user at a measurement opportunity included in a target measurement time zone, and setting, based on the measurement success/failure information in the target measurement time zone, a measurement condition including a standby time from when a gait is sensed at a measurement opportunity included in a related measurement time zone related to the target measurement time zone to when measurement is started.

A program according to an aspect of the present disclosure causes a computer to execute a process of acquiring measurement success/failure information indicating success/failure of measurement of a physical quantity related to a motion of a foot, the measurement being executed by a measurement device installed at a foot portion of a user at a measurement opportunity included in a target measurement time zone, and a process of setting, based on the measurement success/failure information in the target measurement time zone, a measurement condition including a standby time from when a gait is sensed at a measurement opportunity included in a related measurement time zone related to the target measurement time zone to when measurement is started.

### Advantageous Effects of Invention

According to the present disclosure, it is possible to provide a condition setting device and the like capable of setting a measurement condition for performing gait measurement at an appropriate timing while reducing power consumption.

### BRIEF DESCRIPTION OF DRAWINGS

FIG. 1 is a block diagram illustrating an example of a configuration of a gait measurement system according to a first example embodiment.

FIG. 2 is a conceptual diagram illustrating an example in which a measurement device of the gait measurement system according to the first example embodiment is disposed in footwear.

FIG. 3 is a conceptual diagram for describing a relationship between a local coordinate system and a world coordinate system set in the measurement device of the gait measurement system according to the first example embodiment.

FIG. 4 is a conceptual diagram for describing a measurement time zone set in the measurement device of the gait measurement system according to the first example embodiment.

FIG. 5 is a conceptual diagram for describing a measurement opportunity included in a measurement time zone set in the measurement device of the gait measurement system according to the first example embodiment.

FIG. 6 is a conceptual diagram illustrating an example of a timing chart regarding a measurement opportunity included in a measurement time zone set in the measurement device of the gait measurement system according to the first example embodiment.

FIG. 7 is a conceptual diagram for describing a human body surface.

FIG. 8 is a conceptual diagram for describing a gait event.

FIG. 9 is a block diagram illustrating an example of a configuration of a measurement device of the gait measurement system according to the first example embodiment.

FIG. 10 is a block diagram illustrating an example of a configuration of a condition setting unit included in the measurement device of the gait measurement system according to the first example embodiment.

FIG. 11 is a block diagram illustrating an example of a configuration of a data processing device of the gait measurement system according to the first example embodiment.

FIG. 12 is a flowchart for describing an example of an operation of a control unit included in the measurement device of the gait measurement system according to the first example embodiment.

FIG. 13 is a flowchart for describing an example of a measurement process by a control unit included in the measurement device of the gait measurement system according to the first example embodiment.

FIG. 14 is a flowchart for describing an example of a sensor data measurement process by a control unit included in the measurement device of the gait measurement system according to the first example embodiment.

FIG. 15 is a flowchart for describing an example of an operation of a measurement condition setting unit included in the measurement device of the gait measurement system according to the first example embodiment.

FIG. 16 is a flowchart for describing an example of an operation of a data processing device included in the measurement device of the gait measurement system according to the first example embodiment.

FIG. 17 is a block diagram for describing an example of a configuration of a gait measurement system according to the second example embodiment.

FIG. 18 is a block diagram for describing an example of a configuration of a measurement device of the gait measurement system according to a second example embodiment.

FIG. 19 is a block diagram for describing an example of a configuration of a data processing device of the gait measurement system according to the second example embodiment.

FIG. 20 is a flowchart for describing an example of a configuration of a condition setting device according to a third example embodiment.

FIG. 21 is a block diagram illustrating an example of a hardware configuration that implements control and a process of each example embodiment.

### EXAMPLE EMBODIMENT

Hereinafter, embodiments of the present invention will be described with reference to the drawings. However, the example embodiments described below have technically preferable limitations for carrying out the present invention, but the scope of the invention is not limited to the following. In all the drawings used in the following description of the example embodiment, the same reference numerals are given to the same parts unless there is a particular reason. In the following example embodiments, repeated description of similar configurations and operations may be omitted.

### First Example Embodiment

First, a gait measurement system according to a first example embodiment will be described with reference to the drawings. The gait measurement system according to the present example embodiment measures a physical quantity (sensor data) related to a motion of a foot by a measurement device installed at footwear worn by a user. For example, the physical quantity related to a motion of the foot includes acceleration in the three axial directions (also referred to as spatial acceleration) measured by the acceleration sensor, angular velocities around the three axes (also referred to as spatial angular velocities) measured by the angular velocity sensor, and the like. The gait measurement system of the present example embodiment measures the gait of the user based on the measured sensor data.

### (Configuration)

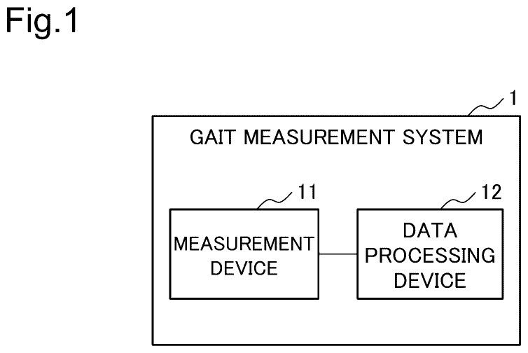

FIG. 1 is a block diagram illustrating a configuration of a gait measurement system 1 of the present example embodiment. The gait measurement system 1 includes a measurement device 11 and a data processing device 12. The measurement device 11 and the data processing device 12 may be connected by wire or wirelessly. The measurement device 11 and the data processing device 12 may be configured by a single device.

The measurement device 11 is installed at the foot portion. For example, the measurement device 11 is installed at footwear such as shoes. In the present example embodiment, an example in which the measurement device 11 is disposed at a position on the back side of the arch of foot will be described. The measurement device 11 includes, for example, an inertial measurement device including an acceleration sensor and an angular velocity sensor. An example of the inertial measurement device is an inertial measurement unit (IMU). The IMU includes the acceleration sensor that measures acceleration in the three axis directions and the angular velocity sensor that measures angular velocities around the three axes. The measurement device 11 may be achieved by an inertial measurement device such as a vertical gyro (VG) or an attitude heading (AHRS). The measurement device 11 may be achieved by a global positioning system/inertial navigation system (GPS/INS). The measurement device 11 includes a microcomputer and a microcontroller that perform a control process and an arithmetic process of the measurement device 11, and a real-time clock (hereinafter, also referred to as a clock) that ticks time. Each of the microcomputer and the microcontroller is also referred to as a control unit (controller).

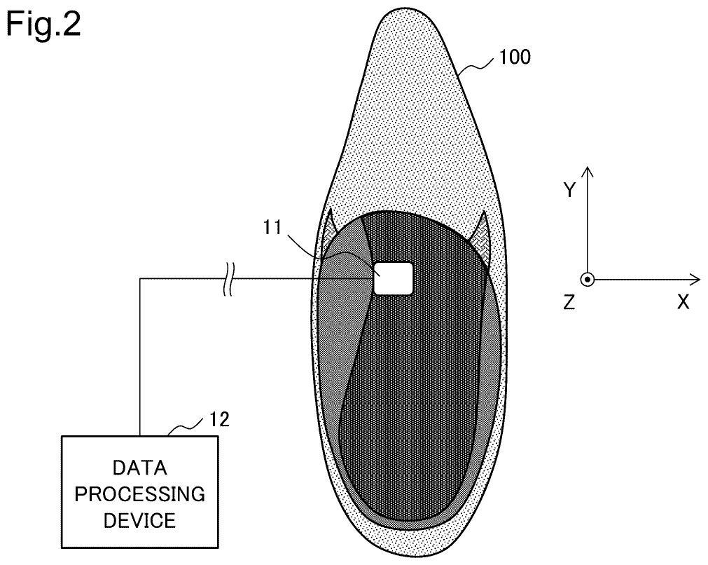

FIG. 2 is a conceptual diagram illustrating an example in which the measurement device 11 is disposed at the shoe 100. In the example of FIG. 2, the measurement device 11 is installed at a position corresponding to the back side of the arch of foot. For example, the measurement device 11 is disposed at an insole inserted into the shoe 100. For example, the measurement device 11 is disposed at the bottom face of the shoe 100. For example, the measurement device 11 is embedded in the main body of the shoe 100. The measurement device 11 may be detachable from the shoe 100 or may not be detachable from the shoe 100.

The measurement device 11 may be disposed at a position other than that of the back side of the arch of foot as long as sensor data related to a motion of the foot can be acquired. The measurement device 11 may be installed at a sock worn by the user or a decorative article such as an anklet worn by the user. The measurement device 11 may be directly attached to the foot or may be embedded in the foot. FIG. 2 illustrates an example in which the measurement device 11 is installed at the shoe 100 for the right foot, but the measurement device 11 may be installed at the shoe 100 for each of the right and left feet. When the measurement device 11 is installed at the shoe 100 for each of the left and right feet, the gait can be measured based on the motion of the left and right feet.

The measurement device 11 includes an acceleration sensor and an angular velocity sensor. The measurement device 11 measures, as a physical quantity related to the motion of the foot of the user wearing the footwear, a physical quantity such as an acceleration in each of the three axis directions (also referred to as a spatial acceleration) measured by the acceleration sensor and an angular velocity around each of the three axes (also referred to as a spatial angular velocity) measured by the angular velocity sensor. The physical quantity related to the motion of the foot measured by the measurement device 11 also includes a speed and a position (trajectory) in each of the three axis directions and an angle around each of the three axes calculated by integrating the acceleration and the angular velocity. The measurement device 11 converts the measured analog data (physical quantity) into digital data (also referred to as sensor data).

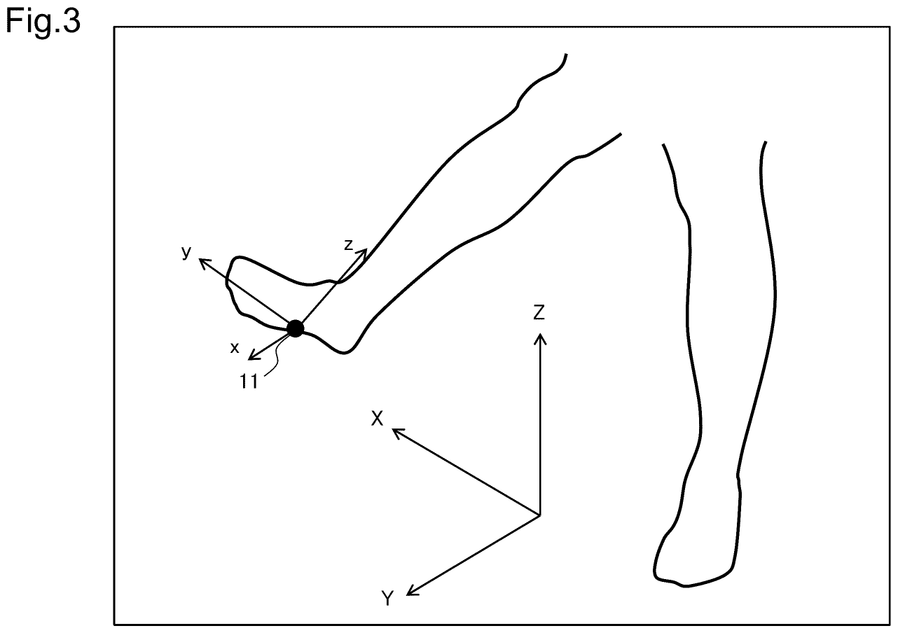

FIG. 3 is a conceptual diagram for describing a local coordinate system (x-axis, y-axis, z-axis) set in the measurement device 11 and a world coordinate system (X-axis, Y-axis, Z-axis) set with respect to the ground in a case where the measurement device 11 is installed at a position corresponding to the back side of the arch of the foot. In the world coordinate system (X-axis, Y-axis, Z-axis), in a state where the user stands upright, the lateral direction of the user is set to the X-axis direction (the rightward direction is positive), the front direction of the user (the traveling direction) is set to the Y-axis direction (the forward direction is positive), and the gravity direction is set to the Z-axis direction (the vertically upward direction is positive). In the present example embodiment, a local coordinate system including an x direction, a y direction, and a z direction based on the measurement device 11 is set.

The measurement device 11 transmits the converted sensor data to the data processing device 12. For example, the measurement device 11 is connected to the data processing device 12 via a mobile terminal (not illustrated) carried by the user. In a case where the communication between the measurement device 11 and the mobile terminal is successful and the sensor data is transmitted from the measurement device 11 to the mobile terminal, the measurement in the measurement time zone is ended. In a case where the communication between the measurement device 11 and the mobile terminal is successful, the clock time of the measurement device 11 is synchronized with the clock time of the mobile terminal. For example, in a case where the communication between the measurement device 11 and the mobile terminal fails and the sensor data is not transmitted from the measurement device 11 to the mobile terminal, the sensor data in the measurement time zone may be retransmitted in the next or subsequent measurement time zone. For example, in a case where the communication between the measurement device 11 and the mobile terminal fails, the transmission of the sensor data in the measurement time zone may be repeated until the communication succeeds. For example, in a case where communication between the measurement device 11 and the mobile terminal fails, transmission of sensor data in the measurement time zone may be repeated within a predetermined time. Sensor data in the measurement time zone in which transmission has failed may be stored in a storage device (not illustrated) such as an electrically erasable programmable read only memory (EEPROM) until the next transmission timing.

In a case where the measurement devices 11 are mounted on both the right and left legs/feet, when the clock times of the measurement devices 11 are synchronized with the time of the mobile terminal, the times of the measurement devices 11 mounted on both the legs/feet can be synchronized. The measurement devices 11 mounted on both feet may perform measurement at the same timing or may perform measurement at different timings. For example, in a case where the measurement timings by the measurement devices 11 mounted on both feet greatly deviate based on the measurement time at both feet and the number times of measurement failures, correction may be performed to reduce the deviation between the measurement timings. The correction of the measurement timing may be performed by the data processing device 12 that can verify the sensor data of both feet or by a higher system.

A mobile terminal (not illustrated) connected to the measurement device 11 is achieved by a communication device that can be carried by a user. For example, the mobile terminal is a portable communication device having a communication function, such as a smartphone, a smart watch, or a mobile phone. The mobile terminal receives sensor data related to the motion of the user's foot from the measurement device 11. The mobile terminal transmits the received sensor data to a server or the like on which the data processing device 12 is mounted. The function of the data processing device 12 may be implemented by application software or the like (also referred to as an application) installed in the mobile terminal. In this case, the mobile terminal processes the received sensor data by application installed therein.

For example, when the use of the gait measurement system 1 of the present example embodiment is started, the application related to the gait measurement system 1 is downloaded to the mobile terminal of the user and the user information is registered. At this time, the device identifier and the measurement time zone of the measurement device 11 are registered in an application of the user's mobile terminal. For example, the measurement time zone registered in the application is transmitted to the measurement device 11 and set in the measurement device 11. For example, when the measurement time zone is set in the measurement device 11, by synchronizing the time of the mobile terminal with the clock time of the measurement device 11, the time unique to the measurement device 11 can be set according to the universal time.

The measurement device 11 is activated in a measurement time zone set in advance. The measurement time zone is set for each user. The measurement time zone includes a time at which measurement is started (also referred to as a measurement time zone start time) and a time during which measurement is continued. The measurement time zone may include a time at which measurement ends (also referred to as a measurement time zone end time). The measurement time zone may be defined by a measurement time zone start time and a measurement time zone end time.

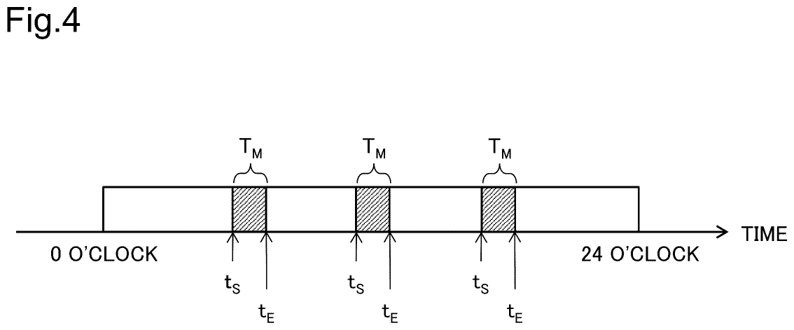

FIG. 4 is a conceptual diagram for describing a measurement time zone. In the example of FIG. 4, three measurement time zones T_{M} are set between 0:00 and 24:00 (one day). The measurement time zone T_{M} is a time zone between a measurement time zone start time t_{S} and a measurement time zone end time t_{E}. For example, each of the three measurement time zones T_{M} is set to the same time width. For example, each of the three measurement time zones T_{M} is set to three hours. For example, each of the three measurement time zones T_{M} may be set to a time width different from each other. For example, the measurement time zone T_{M} is set in accordance with a school commuting time zone or a commuting time zone in the morning and evening of the user. For example, the measurement time zone TM is set in accordance with the user's lunch break time zone. For example, the measurement time zone T_{M} is set in accordance with the time zone of the user's walk. The examples described here are an example, and the measurement time zone start time tₛ, the measurement time zone end time t_{E}, and the time width of the measurement time zone T_{M} can be set to any value. For example, the measurement time zone start time t_{S}, the measurement time zone end time t_{E}, and the time width of the measurement time zone TM may be set in advance at the time of shipment from the factory, may be set when the user starts use, or may be set at any timing by the user.

When the measurement device 11 is activated according to arrival of the measurement time zone, the mode shifts from the sleep mode to the vibration detection mode. In the sleep mode, the operation other than the clock is stopped. For example, in the sleep mode, a current of several microamps (μA) flows in order for the clock to operate. When the mode shifts to the vibration detection mode, the sensor and the control unit are activated with low power consumption. In the vibration detection mode, the sensor and the control unit are controlled to have a minimum current value capable of detecting the vibration at the start of the gait of the user. For example, the current value of the sensor in the vibration detection mode is set to a dozen or so μA.

When detecting the start of the gait in the vibration detection mode, the measurement device 11 shifts to the measurement mode. For example, when the acceleration or the angular velocity exceeds a preset threshold value in the vibration detection mode, the measurement device 11 shifts to the measurement mode. For example, when the acceleration in the traveling direction exceeds 3.5 G in the vibration detection mode, the measurement device 11 shifts to the measurement mode. When the mode shifts to the measurement mode, the limitation of the current value of the sensor is released. For example, when the mode shifts to the measurement mode, the current value of the sensor reaches the order of mA at the maximum. When the mode shifts to the measurement mode, the limitation of the current value of the control unit is released. For example, when the limitation of the current value of the control unit is released, a current of several milliamperes (mA) flows.

When a predetermined time (also referred to as a standby time) elapses after shifting to the measurement mode, measurement of sensor data is started. The standby time is set by the measurement device 11. The timing of measurement that comes when the standby time elapses after the mode shifts to the measurement mode is referred to as a measurement opportunity. For example, several measurement opportunities are set in one measurement time zone.

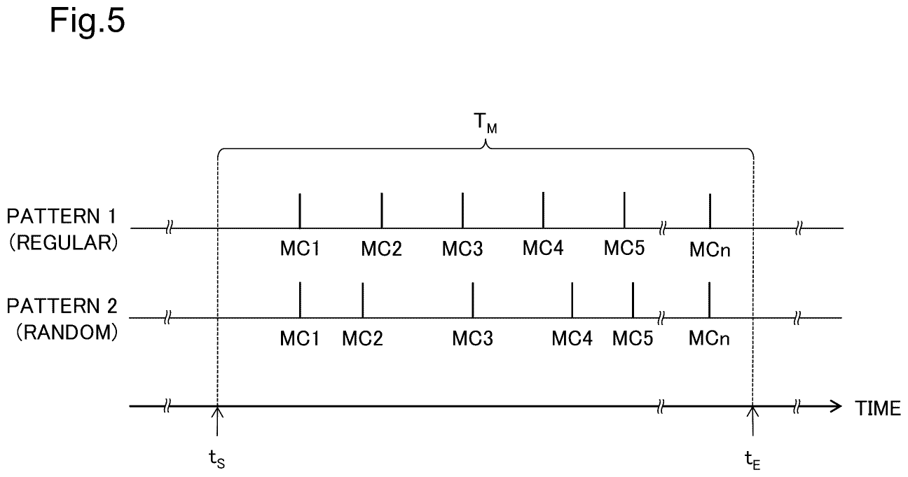

FIG. 5 is a conceptual diagram for describing a measurement opportunity set in the measurement time zone T_{M}. In the example of FIG. 5, a plurality of measurement opportunities (MC1 to MCn) is set in the measurement time zone T_{M} between the measurement time zone start time t_{S} and the measurement time zone end time t_{E} (n is a natural number). The plurality of measurement opportunities (MC1 to MCn) may be set regularly or randomly.

The pattern 1 in FIG. 5 is an example in which a plurality of measurement opportunities (MC1 to MCn) is regularly set. For example, in the pattern 1, the plurality of measurement opportunities (MC1 to MCn) is set at equal intervals within the period of the measurement time zone TM. For example, in the pattern 1, the plurality of measurement opportunities (MC1 to MCn) is set at intervals based on a preset rule.

The pattern 2 in FIG. 5 is an example in which a plurality of measurement opportunities (MC1 to MCn) is randomly set. For example, in the pattern 2, a plurality of measurement opportunities (MC1 to MCn) is randomly set within the period of the measurement time zone T_{M}. For example, in the pattern 2, the plurality of measurement opportunities (MC1 to MCn) may be set at predetermined random intervals, or may be set at random according to arrival of the measurement time zone.

The standby time from the measurement start time is preferably set in accordance with the time from the gait detection to the stable gait. For example, assuming a commuting time zone, the gait of the user is different depending on the time of getting on and off an elevator in the residence, the time of the gait from the home to a bus stop or a station, whether to stop at a convenience store, and the like. For example, when a plurality of measurement opportunities (MC1 to MCn) arrives regularly, the measurement may continue to deviate similarly from the period of the stable gait, and the measurement failure may be continued. On the other hand, when a plurality of measurement opportunities (MC1 to MCn) is randomly set, measurement is performed at timing deviating from specific regularity, and thus, there is a possibility that the measurement timing matches the period of the stable gait and the measurement probability is improved.

When the mode shifts to the measurement mode, the digit of the current value used in the measurement device 11 increases from the order of μA to the order of mA. Therefore, the measurement mode is preferably set as short as possible. That is, setting the measurement opportunity to which timing in one measurement time zone is a key to reduce power consumption. In the measurement time zone, the measurement may be successful even once. For example, when the measurement can be performed once in the measurement time zone, the measurement in the measurement time zone is ended. On the other hand, in a case where the measurement fails in all the measurement opportunities included in the measurement time zone, the measurement in the measurement time zone is regarded as a failure. For example, the measurement may be performed several times in the measurement time zone, and the measurement values in the measurement time zone may be averaged. However, the power consumption can be reduced by ending the measurement with one success rather than repeating the measurement several times.

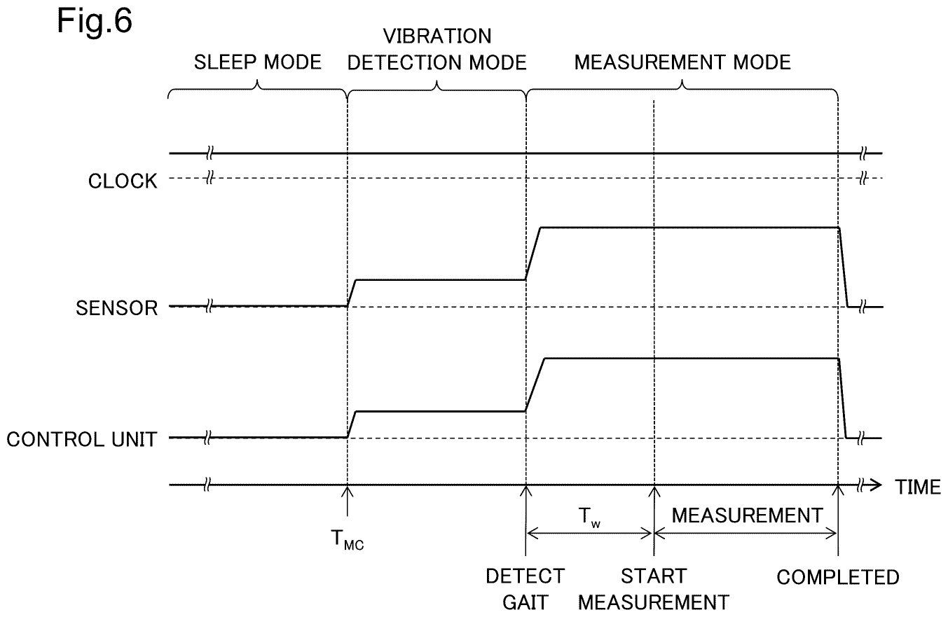

FIG. 6 is a timing chart for describing an example of operation timings of a clock, a sensor, and a control unit included in the measurement device 11. The timing chart of FIG. 6 schematically illustrates the presence or absence of the operation and the magnitude of the power consumption during the operation, but does not indicate accurate values. The clock constantly operates during the use of the measurement device 11. In FIG. 6, a situation in which the clock ticks time is omitted. When the clock time reaches the time of the measurement opportunity (measurement start time T_{MC}), the sensor and the control unit are activated to shift from the sleep mode to the vibration detection mode. In the vibration detection mode, the sensor and the control unit operate with low power consumption with which the gait can be detected. When the gait is detected by the sensor, the mode shifts to the measurement mode. When shifting to the measurement mode, the control unit operates with normal power consumption. In the normal power consumption, the control unit releases the limitation of the current value. The control unit may be configured to be activated at the stage of shifting to the measurement mode.

In FIG. 6, when the standby time T_{w} elapses from the gait detection, the measurement of the sensor data by the control unit is started. When the measurement at the measurement opportunity succeeds, the measurement in the measurement time zone is completed. In a case where the measurement at the measurement opportunity fails, the process waits until the next measurement opportunity. When all the measurement opportunities in the measurement time zone end, the measurement in the measurement time zone ends. In the present example embodiment, in order to suppress the occurrence of wasteful power consumption, the measurement in the measurement time zone is completed when all the measurement opportunities in the measurement time zone have ended, regardless of whether the measurement succeeds. For example, in a case where the measurement fails in a certain measurement time zone, the measurement may be continued for a predetermined period from the end of the measurement time zone until the measurement succeeds.

For example, the measurement time at each measurement opportunity is set to about 10 seconds. In the case of the gait for about 10 seconds, sensor data for about three walks is measured. The measurement device 11 transmits sensor data measured in the measurement time zone to the data processing device 12. For example, the measurement device 11 may transmit the sensor data for several walks as it is to the data processing device 12, or may average the sensor data for several walks and then transmit the averaged sensor data to the data processing device 12. For example, the measurement device 11 may extract sensor data for one walk from sensor data for several walks to transmit the extracted sensor data for one walk to the data processing device 12. By averaging sensor data for several walks or extracting sensor data for one walk, the amount of data to be transmitted can be reduced. For example, in a case where transmission of sensor data measured in a certain measurement time zone fails, the sensor data whose transmission has failed is primarily stored in a storage unit (not illustrated). Then, a plurality of times of sensor data may be collectively transmitted at a transmission timing of the sensor data in the next measurement time zone.

The measurement device 11 records information (also referred to as measurement success/failure information) regarding whether the measurement in the measurement time zone succeeds. For example, the measurement success/failure information includes information such as a measurement time, an activation condition, information indicating at which measurement opportunity the measurement has succeeded (also referred to as trial count information), and a timing at which a measurement failure has occurred. For example, the measurement success/failure information may include information such as a time zone, a date and time, a day of the week, and a season in which the measurement failure occurs. For example, the measurement success/failure information may include a flag indicating the success/failure of the measurement. For example, the measurement success/failure information may include sensor data. According to measurement success/failure information in a certain measurement time zone (also referred to as a control measurement time zone), the measurement device 11 sets a standby time in a measurement time zone (also referred to as a related measurement time zone) related to the certain measurement time zone. Details of setting of the standby time by the measurement device 11 will be described later.

The data processing device 12 acquires sensor data from the measurement device 11. The data processing device 12 generates a waveform (also referred to as a gait waveform) based on the time-series data of the acquired sensor data. For example, the data processing device 12 generates a gait waveform related to an acceleration, an angular velocity, a velocity, an angle, and a position (trajectory) on a face (also referred to as a human body surface) set for the human body.

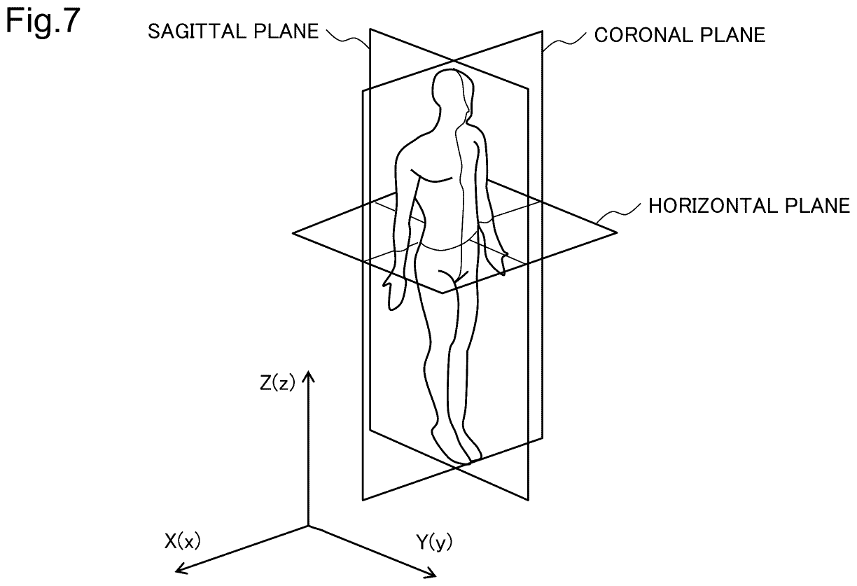

FIG. 7 is a conceptual diagram for describing a human body surface. In the present example embodiment, a sagittal plane dividing the body into left and right, a coronal plane dividing the body into front and rear, and a horizontal plane dividing the body horizontally are defined. In the upright state as illustrated in FIG. 7, the world coordinate system coincides with the local coordinate system. In the present example embodiment, rotation in the sagittal plane with the x-axis as a rotation axis is defined as roll, rotation in the coronal plane with the y-axis as a rotation axis is defined as pitch, and rotation in the horizontal plane with the z-axis as a rotation axis is defined as yaw. A rotation angle in a sagittal plane with the x-axis as a rotation axis is defined as a roll angle, a rotation angle in a coronal plane with the y-axis as a rotation axis is defined as a pitch angle, and a rotation angle in a horizontal plane with the z-axis as a rotation axis is defined as a yaw angle.

The data processing device 12 executes a data process related to gait measurement using the generated gait waveform. For example, the data processing device 12 detects a gait event using a gait waveform. For example, the data processing device 12 estimates the physical characteristics and physical condition of the user based on the sensed gait event. The data processing by the data processing device 12 is not particularly limited as long as it is data processing related to the gait.

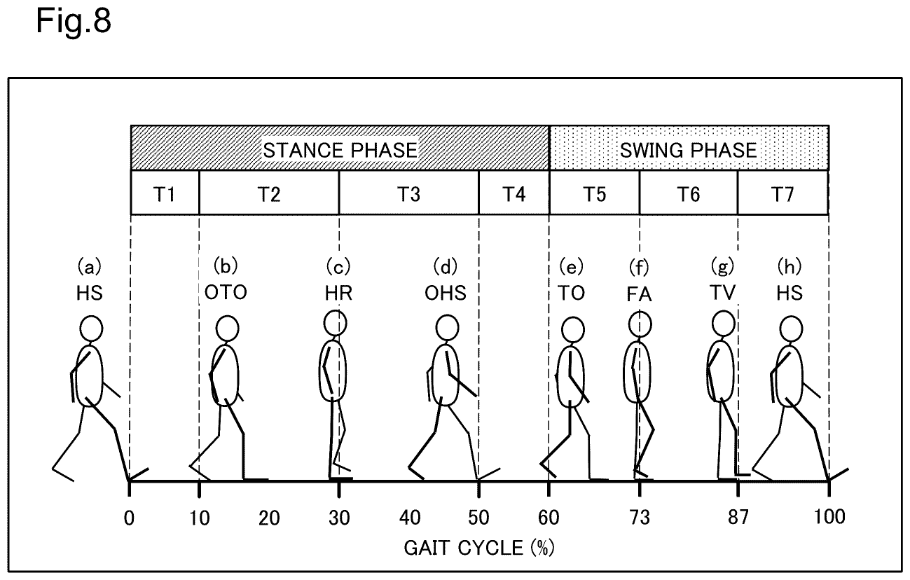

FIG. 8 is a conceptual diagram for describing one gait cycle with the right foot as a reference. The horizontal axis of FIG. 8 is a normalized gait cycle with one gait cycle of the right foot as 100% with a time point at which the heel of the right foot lands on the ground as a starting point and a time point at which the heel of the right foot lands on the ground next as an ending point. The one gait cycle of one foot is roughly divided into a stance phase in which at least part of the back side of the foot is in contact with the ground and a swing phase in which the back side of the foot is away from the ground. In the present example embodiment, normalization is performed in such a way that the stance phase occupies 60% and the swing phase occupies 40%. The stance phase is further subdivided into an initial stance period T1, a mid-stance period T2 of standing, a terminal stance period T3 of standing, and a pre-swing period T4. The swing phase is further subdivided into an initial swing period T5, a mid-swing period T6, and a terminal swing period T7. In the gait waveform in one gait cycle, the time point when the heel lands on the ground may not be set as a starting point.

FIG. 8(a) represents an event (heel strike (HS)) in which the heel of the right foot is grounded. FIG. 8(b) represents an event (opposite toe off (OTO)) in which the toe of the left foot moves away from the ground in a state where the ground contact surface of the sole of the right foot is in contact with the ground. FIG. 8(c) represents an event (heel rise (HR)) in which the heel of the right foot is lifted in a state where the ground contact surface of the sole of the right foot is in contact with the ground. FIG. 8(d) represents an event (opposite heel strike (OHS)) in which the heel of the left foot is grounded. FIG. 8(e) represents an event (toe off (TO)) in which the toe of the right foot is away from the ground in a state where the ground contact surface of the sole of the left foot is in contact with the ground. FIG. 8(f) represents an event (foot adjacent (FA)) in which the left foot and the right foot intersect with each other in a state where the ground contact surface of the sole of the left foot is in contact with the ground. FIG. 8(g) represents an event (tibia vertical (TV)) in which the tibia of the left foot is substantially perpendicular to the ground in a state where the sole of the right foot is in contact with the ground. FIG. 8(h) represents an event (heel strike (HS)) in which the heel of the right foot is grounded. FIG. 8(h) corresponds to the ending point of the gait cycle starting from FIG. 8(a) and corresponds to the starting point of the next gait cycle.

For example, the data processing device 12 is mounted on a server (not illustrated) or the like. For example, the data processing device 12 may be achieved by an application server. For example, the data processing device 12 may be achieved by an application installed in a mobile terminal (not illustrated). For example, a result of data processing by the data processing device 12 is displayed on a screen of a display device (not illustrated). For example, a result of data processing by the data processing device 12 is output to a system that uses the result. The use of the result of the data processing by the data processing device 12 is not particularly limited.

### [Measurement Device]

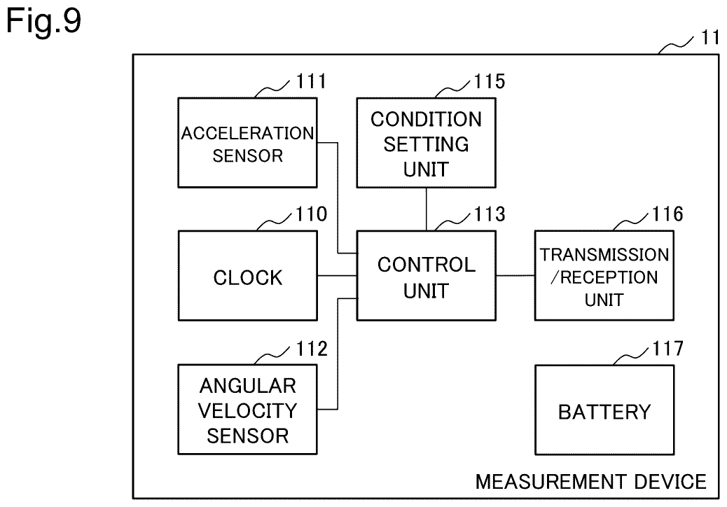

Next, a detailed configuration of the measurement device 11 will be described with reference to the drawings. FIG. 9 is a block diagram illustrating an example of a detailed configuration of the measurement device 11. The measurement device 11 includes a clock 110, an acceleration sensor 111, an angular velocity sensor 112, a control unit 113, a condition setting unit 115, a transmission/reception unit 116, and a battery 117. The condition setting unit 115 may be configured as a single device (condition setting device).

The clock 110 is a real-time clock that ticks time at a predetermined cycle. For example, the clock 110 includes a crystal oscillator. The time that the clock 110 ticks is synchronized with a clock time included in a mobile terminal (not illustrated) or the like on which the data processing device 12 is mounted at a timing when the measurement device 11 and the data processing device 12 are connected. The clock 110 continues to operate in any of the sleep mode, the vibration detection mode, and the measurement mode. The time that the clock 110 ticks is referred to at the time of control by the control unit 113 or switching of the mode.

The acceleration sensor 111 is a sensor that measures acceleration (also referred to as spatial acceleration) in the three axial directions. The acceleration sensor 111 is activated when the time that the clock 110 ticks reaches the measurement start time, and shifts to the vibration detection mode. In the vibration detection mode, the acceleration sensor 111 operates in a low power consumption mode in which gait detection is possible. For example, the current value of the acceleration sensor 111 in the vibration detection mode is set to a dozen or so μA. When the start of the gait is detected in the vibration detection mode, the acceleration sensor 111 shifts to the measurement mode. For example, when the acceleration exceeds a preset threshold value in the vibration detection mode, the acceleration sensor 111 shifts to the measurement mode. For example, when the acceleration in the traveling direction exceeds 3.5 G in the vibration detection mode, the measurement device 11 shifts to the measurement mode. In a case where the gait is detected based on the angular velocity measured by the angular velocity sensor 112, the acceleration sensor 111 may be configured to be activated at the timing of shifting to the measurement mode. When the mode shifts to the measurement mode, the limitation of the current value of the acceleration sensor 111 is released. For example, when shifting to the measurement mode, the current value of the acceleration sensor 111 reaches the order of mA at the maximum. In the measurement mode, the acceleration sensor 111 measures the spatial acceleration. The acceleration sensor 111 outputs the measured acceleration to the control unit 113.

For example, a sensor of a piezoelectric type, a piezoresistive type, a capacitance type, or the like can be used as the acceleration sensor 111. As long as the sensor used for the acceleration sensor 111 can measure an acceleration, the measurement method is not limited.

The angular velocity sensor 112 is a sensor that measures angular velocities in the three axial directions (also referred to as spatial angular velocities). The angular velocity sensor 112 is activated when the time that the clock 110 ticks reaches the measurement start time, and shifts to the vibration detection mode. In the vibration detection mode, the angular velocity sensor 112 operates in a low power consumption mode in which gait detection is possible. For example, the current value of angular velocity sensor 112 in the vibration detection mode is set to a dozen or so μA. When the start of the gait is detected in the vibration detection mode, the angular velocity sensor 112 shifts to the measurement mode. For example, when the acceleration exceeds a preset threshold value in the vibration detection mode, the angular velocity sensor 112 shifts to the measurement mode. In a case where the gait is detected based on the acceleration measured by the acceleration sensor 111, the angular velocity sensor 112 may be configured to be activated at the timing of shifting to the measurement mode. When shifting to the measurement mode, the limitation of the current value of the angular velocity sensor 112 is released. For example, when shifting to the measurement mode, the current value of the acceleration sensor 111 reaches the order of mA at the maximum. In the measurement mode, the angular velocity sensor 112 measures the spatial angular velocity. Angular velocity sensor 112 outputs the measured angular velocity to control unit 113.

For example, a sensor of a vibration type, a capacitance type, or the like can be used as the angular velocity sensor 112. As long as the sensor used for the angular velocity sensor 112 can measure an angular velocity, the measurement method is not limited.

The control unit 113 is activated when shifting to the vibration detection mode. In the vibration detection mode, the control unit 113 operates in a low power consumption mode in which the gait can be detected based on measurement values measured by the acceleration sensor 111 and the angular velocity sensor 112. When the start of the gait is detected in the vibration detection mode, the control unit 113 shifts to the measurement mode. For example, in the vibration detection mode, when the acceleration measured by the acceleration sensor 111 exceeds a preset threshold value, the control unit 113 shifts to the measurement mode. For example, in the vibration detection mode, when the acceleration in the traveling direction measured by the acceleration sensor 111 exceeds 3.5 G, the control unit 113 shifts to the measurement mode. The control unit 113 may be configured to be activated at the stage of shifting to the measurement mode.

In the measurement mode, the control unit 113 measures the sensor data under a normal operating condition based on the set measurement condition. For example, when shifting to the measurement mode, the control unit 113 operates by switching from an operating condition of low power consumption to a normal operating condition. For example, a current of several milliamperes (mA) flows during the normal operation of the control unit. For example, the control unit 113 switches to the normal operating condition at the timing when the standby time has elapsed from the start time of the measurement mode. The control unit 113 acquires the accelerations in the three axial directions and the angular velocities around the three axes from the acceleration sensor 111 and the angular velocity sensor 112, respectively.

The control unit 113 converts the acquired acceleration and angular velocity into digital data to output the converted digital data (also referred to as sensor data) to the transmission/reception unit 116. The sensor data includes at least acceleration data converted into digital data and angular velocity data converted into digital data. The sensor data includes measurement times of the acceleration and the angular velocity. The acceleration data includes acceleration vectors in the three axial directions. The angular velocity data includes angular velocity vectors around three axes. The acquisition times of the acceleration data and the angular velocity data are associated with the acceleration data and the angular velocity data. The control unit 113 may be configured to output sensor data obtained by adding correction such as a mounting error, temperature correction, and linearity correction to the acquired acceleration data and angular velocity data. The control unit 113 may generate angle data around the three axes using the acquired acceleration data and angular velocity data.

The control unit 113 outputs measurement success/failure information of the measured sensor data to a measurement condition setting unit 156. The measurement success/failure information output to the measurement condition setting unit 156 is used for generating the standby time in the measurement mode.

For example, the control unit 113 is a microcomputer or a microcontroller that performs overall control and a data process of the measurement device 11. For example, the control unit 113 includes a central processing unit (CPU), a random access memory (RAM), a read only memory (ROM), a flash memory, and the like. The control unit 113 controls the acceleration sensor 111 and the angular velocity sensor 112 to measure the angular velocity and the acceleration. For example, the control unit 113 performs analog-to-digital conversion (AD conversion) on physical quantities (analog data) such as the measured angular velocity and acceleration, and stores the converted digital data in the flash memory. The physical quantity (analog data) measured by each of the acceleration sensor 111 and the angular velocity sensor 112 may be converted into digital data in each of the acceleration sensor 111 and the angular velocity sensor 112. The digital data stored in the flash memory is output to the transmission/reception unit 116 at a predetermined timing.

The condition setting unit 115 acquires the measurement success/failure information in the measurement time zone from the control unit 113. The condition setting unit 115 records the acquired measurement success/failure information. The measurement success/failure information includes information such as a measurement time, an activation condition, and trial count information indicating at which measurement opportunity the measurement was successful. The measurement success/failure information may include sensor data. The measurement success/failure information may include a flag (also referred to as a success flag) in a case where the measurement succeeds in the measurement time zone and a flag (also referred to as a failure flag) in a case where the measurement fails in the measurement time zone. The measurement success/failure information to which the success flag is attached is also referred to as success information, and the measurement success/failure information to which the failure flag is attached is also referred to as failure information. The condition setting unit 115 registers, in a storage unit (not illustrated), failure information including a date and time of a measurement time zone in a case where measurement has been executed but the measurement has not been completed and the number times of the failure in the measurement time zone.

The condition setting unit 115 sets the standby time in the measurement time zone related to the measurement time zone according to the measurement success/failure information for each measurement time zone. The standby time set based on the measurement success/failure information in a certain measurement time zone may be reflected in a measurement time zone immediately after the measurement time zone, or may be reflected in a measurement time zone on or after the next day. In order to set the standby time according to the user's habit, it is preferable that the standby time is reflected in the same measurement time zone on or after the next day.

The condition setting unit 115 calculates a probability of success or a probability of failure in measurement for each measurement time zone. Hereinafter, an example in which the standby time is set based on the probability of success in measurement will be described. The condition setting unit 115 extracts a measurement time zone having a low probability of success. The measurement time zone having a low probability of success is a time zone in which the standby time is to be changed (also referred to as a target time zone). The condition setting unit 115 extracts a failure time zone in the extracted target time zone from failure information accumulated over several weeks. For example, the condition setting unit 115 extracts failure information in the same time zone on the same day of the week. The condition setting unit 115 changes the standby time in the target time zone based on the extracted failure information. For example, the condition setting unit 115 changes the standby time in the target time zone within a predetermined time range such as ±5 to 10 seconds. The standby time may be changed regularly or randomly.

The condition setting unit 115 calculates a probability of success or a probability of failure in measurement in the target time zone in which the standby time has been changed. In a case where the probability of success or the probability of failure in measurement in the target time zone satisfies the criterion, the condition setting unit 115 sets the standby time in the target time zone as it is. For example, in a case where the probability of success in measurement in the target time zone exceeds a predetermined threshold value or in a case where the probability of failure in measurement in the target time zone falls below a predetermined threshold value, the condition setting unit 115 does not change the standby time in the target time zone any more.

In a case where the probability of success or the probability of failure in measurement in the target time zone does not satisfy the criterion, the condition setting unit 115 changes the standby time in the target time zone again. For example, in a case where an increase in the probability of success in measurement or a decrease in the probability of failure in measurement in the target time zone is insufficient as a result of increasing the standby time, the condition setting unit 115 further increases the standby time. For example, in a case where an increase in the probability of success in measurement or a decrease in the probability of failure in measurement in the target time zone is insufficient as a result of shortening the standby time, the condition setting unit 115 further shortens the standby time. For example, in a case where a decrease in the probability of success in measurement or an increase in the probability of failure in measurement occurs in the target time zone as a result of increasing the standby time, the condition setting unit 115 shortens the standby time. For example, in a case where a decrease in the probability of success in measurement or an increase in the probability of failure in measurement occurs in the target time zone as a result of shortening the standby time, the condition setting unit 115 lengthens the standby time.

For example, in a case where a measurement failure occurs at a measurement opportunity included in a certain measurement time zone, the standby time for the next measurement opportunity of the measurement time zone may be changed. In this way, there is a possibility that the period during which the user performs a stable gait coincides with that of the measurement opportunity and the measurement succeeds. In a case where the measurement is successful, when the standby time in this case is set to the standby time of the same measurement time zone of the next day, there is a high possibility that the measurement in the same measurement time zone of the next day will be successful. In a case where the measurement is not successful even when the standby time is shifted many times, the measurement time zone may be shifted by about one hour. The standby time for each measurement time zone is fixed at a time when the probability of success in measurement is high.

The transmission/reception unit 116 acquires sensor data from the control unit 113. The transmission/reception unit 116 transmits the acquired sensor data to the data processing device 12. The transmission/reception unit 116 may transmit the sensor data to the data processing device 12 via a wire such as a cable, or may transmit the sensor data to the data processing device 12 via wireless communication. For example, the transmission/reception unit 116 is configured to transmit sensor data to the data processing device 12 via a wireless communication function (not illustrated) conforming to a standard such as Bluetooth (registered trademark) or WiFi (registered trademark). The communication function of the transmission/reception unit 116 may conform to a standard other than Bluetooth (registered trademark) or WiFi (registered trademark).

The battery 117 is a power source for components included in the measurement device 11. The power of the battery 117 is consumed in the operations of the clock 110, the acceleration sensor 111, the angular velocity sensor 112, the control unit 113, the condition setting unit 115, and the transmission/reception unit 116. For example, the battery 117 is achieved by a primary battery. For example, the battery 117 is achieved by a primary battery such as a lithium battery, a nickel-based battery, a manganese-based battery, an alkaline battery, a nickel-manganese battery, a silver oxide battery, a mercury battery, or an air-zinc battery. For example, the battery 117 may be achieved by a secondary battery. For example, it may be achieved by a secondary battery such as a lithium-ion battery or a nickel-metal hydride battery. In a case where the measurement device 11 is mounted at the insole inserted into the footwear as in the present example embodiment, the battery 117 preferably has a thin structure like a button battery. For example, the battery 117 may be achieved by a button type battery such as a graphite fluoride lithium battery, a lithium manganese dioxide battery, a copper oxide lithium battery, an alkaline battery, a mercury battery, an air-zinc battery, a silver oxide battery, or a nickel-metal hydride battery. The type and form of the battery 117 are not limited as long as it can be used for gait measurement.

### [Condition Setting Unit]

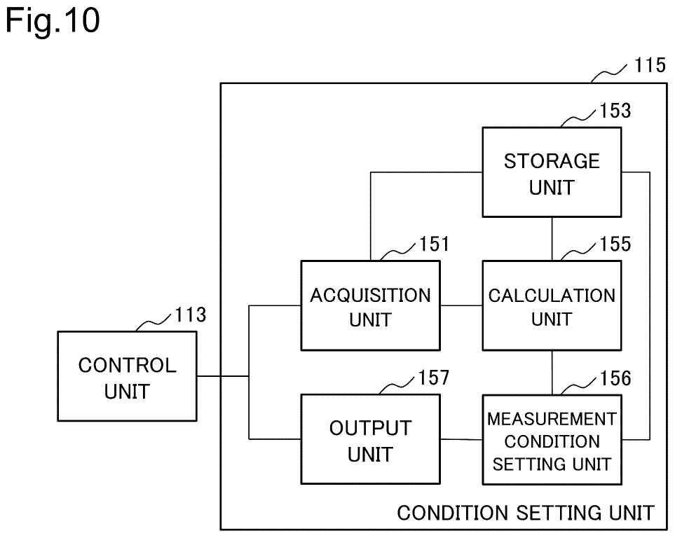

Next, a detailed configuration of the condition setting unit 115 included in the measurement device 11 will be described with reference to the drawings. FIG. 10 is a block diagram illustrating an example of a configuration of the condition setting unit 115. The condition setting unit 115 includes an acquisition unit 151, a storage unit 153, a measurement condition setting unit 156, a calculation unit 155, and an output unit 157. The condition setting unit 115 may be configured as a single device.

The acquisition unit 151 acquires the measurement success/failure information in the measurement time zone from the control unit 113. The acquisition unit 151 stores the acquired measurement success/failure information in the storage unit 153. For example, the measurement success/failure information includes information such as a measurement time, an activation condition, information indicating at which measurement opportunity the measurement has succeeded (also referred to as trial count information), and a timing at which a measurement failure has occurred. For example, the measurement success/failure information may include information such as a time zone, a date and time, a day of the week, and a season in which the measurement failure occurs. In the measurement success/failure information, a factor related to a measurement failure is also referred to as a failure factor. The measurement success/failure information may include a success flag in a case where the measurement succeeds in the measurement time zone and a failure flag in a case where the measurement fails in the measurement time zone. The measurement success/failure information to which the success flag is attached is also referred to as success information, and the measurement success/failure information to which the failure flag is attached is also referred to as failure information. The measurement success/failure information may include sensor data. The measurement device 11 stores, in the storage unit 153, failure information including the date and time of the measurement time zone in a case where the measurement has been executed but the measurement has not been completed and the number times of the failure in the measurement time zone. The acquisition unit 151 may be configured as the same communication interface as the output unit 157.

The storage unit 153 stores the measurement success/failure information acquired by the acquisition unit 151. The measurement success/failure information includes information such as a measurement time, an activation condition, and trial count information. The measurement success/failure information may include sensor data. The measurement success/failure information may include a success flag in a case where the measurement succeeds in the measurement time zone and a failure flag in a case where the measurement fails in the measurement time zone. The measurement success/failure information to which the success flag is attached is also referred to as success information, and the measurement success/failure information to which the failure flag is attached is also referred to as failure information. For example, the storage unit 153 stores failure information including a date and time of a measurement time zone in a case where measurement has been executed but the measurement has not been completed, and the number times of the failure in the measurement time zone.

The calculation unit 155 calculates a probability of success and a probability of failure in measurement for each measurement time zone based on the measurement success/failure information stored in the storage unit 153. The calculation unit 155 calculates a probability of success and a probability of failure in measurement in the target time zone in which the standby time has been changed. The probability of success and the probability of failure in measurement calculated by the calculation unit 155 are referred to in setting of the standby time by the measurement condition setting unit 156. In the setting of the standby time by the measurement condition setting unit 156, in a case where the probability of success and the probability of failure in measurement are not referred to, the calculation unit 155 may be omitted.

The measurement condition setting unit 156 extracts a measurement time zone with a low probability of success or a measurement time zone with a high probability of failure based on the calculation result by the calculation unit 155. A measurement time zone with a low probability of success or a measurement time zone with a high probability of failure is a time zone (also referred to as a target time zone) in which the standby time is to be changed. That is, the measurement condition setting unit 156 sets the standby time in the measurement time zone related to the target time zone based on the failure information extracted in the target time zone. For example, the measurement condition setting unit 156 changes the standby time in the target time zone within a predetermined time range such as ±5 to 10 seconds. The standby time may be changed regularly or randomly. The measurement condition setting unit 156 sets a measurement condition including the set standby time. The measurement condition setting unit 156 outputs the set measurement condition to the output unit 157.

For example, the measurement condition setting unit 156 extracts a measurement time zone with a low probability of success or a measurement time zone with a high probability of failure based on the calculation result by the calculation unit 155. For example, the measurement condition setting unit 156 extracts a failure time zone in the extracted target time zone from failure information accumulated over several weeks. For example, the measurement condition setting unit 156 extracts failure information in the same time zone on the same day of the week. For example, in the same time zone of the same day of the week, the user is likely to take a similar action. Therefore, when the standby time is set based on the failure information in the same time zone of the same day of the week, the measurement failure is less likely to occur.

In a case where the probability of success or the probability of failure in measurement in the target time zone satisfies the criterion, the measurement condition setting unit 156 does not change the standby time in the target time zone. For example, in a case where the probability of success in measurement in the target time zone exceeds a predetermined threshold value or in a case where the probability of failure in measurement in the target time zone falls below a predetermined threshold value, the measurement condition setting unit 156 does not change the standby time in the target time zone any more.

In a case where the probability of success or the probability of failure measurement in the target time zone does not satisfy the criterion, the measurement condition setting unit 156 changes the standby time in the target time zone again. For example, in a case where an increase in the probability of success or a decrease in the probability of failure in the target time zone is insufficient as a result of increasing the in measurement standby time, the measurement condition setting unit 156 further increases the standby time. For example, in a case where the increase in the probability of success or the decrease in the probability of failure in measurement in the target time zone is insufficient as a result of shortening the standby time, the measurement condition setting unit 156 further shortens the standby time. For example, in a case where a decrease in the probability of success in measurement or an increase in probability of failure in measurement in the target time zone occurs as a result of increasing the standby time, the measurement condition setting unit 156 shortens the standby time. For example, in a case where a decrease in the probability of success in measurement or an increase in probability of failure in measurement in the target time zone occurs as a result of shortening the standby time, the measurement condition setting unit 156 lengthens the standby time.

For example, the measurement condition setting unit 156 sets the standby time based on failure factors such as the number of times of measurement failure and the timing when the failure occurs. For example, the measurement condition setting unit 156 changes the standby time in the target time zone a plurality of times, and selects the standby time in which the probability of success in measurement is maximum. For example, the measurement condition setting unit 156 repeats updating of the standby time until the probability of success in measurement satisfies the threshold value. For example, the measurement condition setting unit 156 may randomly set the standby time in each of a plurality of measurement opportunities included in the same measurement time zone, and set the standby time having a high probability of success. For example, the measurement condition setting unit 156 may set a standby time with a high probability of success in measurement in a certain measurement time zone as a standby time in another measurement time zone.

For example, the measurement condition setting unit 156 may determine a factor of measurement failure by weighting with a month, a week, a time, the number of times, or the like, and set the standby time according to the month, the week, the time, the number of times. For example, the measurement condition setting unit 156 may record the optimal standby time for each day of the week or time and set the standby time according to the day of the week or time. For example, the measurement condition setting unit 156 may reset the standby time in accordance with the change of seasons.

The output unit 157 acquires the measurement condition from the measurement condition setting unit 156. The output unit 157 outputs the measurement condition set by the measurement condition setting unit 156 to the control unit 113. The output unit 157 may be configured as the same communication interface as the acquisition unit 151.

### [Data Processing Device]

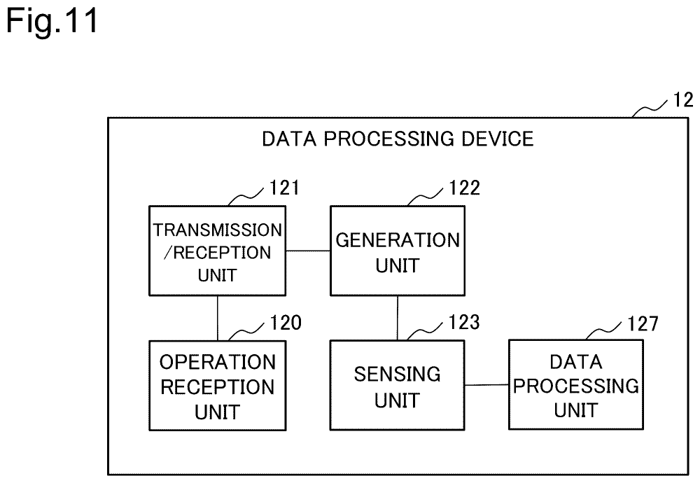

Next, a detailed configuration of the data processing device 12 included in the gait measurement system 1 will be described with reference to the drawings. FIG. 11 is a block diagram illustrating an example of a configuration of the data processing device 12. The data processing device 12 includes an operation reception unit 120, a transmission/reception unit 121, a generation unit 122, a sensing unit 123, and a data processing unit 127.

The operation reception unit 120 receives information (also referred to as input information) input by the user. For example, the input information is input via a terminal device (not illustrated) connected to the data processing device 12 or a mobile terminal (not illustrated) in which the data processing device is installed. For example, the input information is input according to a key input by a keyboard, an input operation on a touch panel, or an operation of a mouse. For example, when the user starts using the gait measurement system 1, input information such as a device identifier of the measurement device 11 and a measurement time zone is input to the operation reception unit 120. The operation reception unit 120 outputs input information to the transmission/reception unit 121.

The transmission/reception unit 121 receives sensor data from the measurement device 11. The transmission/reception unit 121 outputs the received sensor data to the generation unit 122. For example, the transmission/reception unit 121 receives sensor data from the measurement device 11 via wireless communication. For example, the transmission/reception unit 121 is configured to receive sensor data from the measurement device 11 via a wireless communication function (not illustrated) conforming to a standard such as Bluetooth (registered trademark) or WiFi (registered trademark). The communication function of the transmission/reception unit 121 may conform to a standard other than Bluetooth (registered trademark) or WiFi (registered trademark). For example, the transmission/reception unit 121 may receive the sensor data from the measurement device 11 via a wire such as a cable.

The transmission/reception unit 121 acquires input information related to an operation by the user from the operation reception unit 120. The transmission/reception unit 121 transmits the input information to the measurement device 11. For example, the measurement time zone input by the user is set in the measurement device 11. For example, the transmission/reception unit 121 transmits the time of the mobile terminal to the measurement device 11 at a timing when the measurement time zone is set in the measurement device 11, and synchronizes the time of the mobile terminal with the clock time of the measurement device 11. By synchronizing the time of the mobile terminal with the time of the clock of the measurement device 11, the time of the measurement device 11 in which the unique time axis is set can be set in accordance with the time of the universal mobile terminal.

The generation unit 122 acquires sensor data from the transmission/reception unit 121. The generation unit 122 converts the coordinate system of the acquired sensor data from the local coordinate system to the world coordinate system. The generation unit 122 generates time-series data (also referred to as a gait waveform) of the sensor data after conversion into the world coordinate system. The generation unit 122 outputs the generated gait waveform to the sensing unit 123.

For example, the generation unit 122 generates a gait waveform such as a spatial acceleration or a spatial angular velocity. The generation unit 122 integrates the spatial acceleration and the spatial angular velocity, and generates a gait waveform such as the spatial velocity and the spatial angle (plantar angle). The generation unit 122 performs second-order integration of the spatial acceleration to generate a gait waveform of the spatial trajectory. The generation unit 122 generates a gait waveform at a predetermined timing or time interval set in accordance with a general gait cycle or a gait cycle unique to the user. The timing at which the generation unit 122 generates the gait waveform can be set to any timing. For example, the generation unit 122 is configured to continue to generate the gait waveform during a period in which the gait of the user is continued. The generation unit 122 may be configured to generate a gait waveform at a specific time.

The sensing unit 123 acquires the gait waveform from the generation unit 122. The sensing unit 123 detects a gait event from the gait waveform. For example, the sensing unit 123 detects gait events such as heel strike, toe off, foot adjacent, and tibia vertical. The sensing unit 123 outputs, to the data processing unit 127, data used for gait measurement such as the timing of the sensed gait event and a value of sensor data in a predetermined period with the timing of the gait event as the starting point.

The data processing unit 127 acquires data used for gait measurement from the sensing unit 123. The data processing unit 127 performs gait measurement using the acquired data. For example, the data processing unit 127 estimates the physical characteristics and the physical condition of the user based on the sensed gait event. The data processing by the data processing unit 127 is not particularly limited as long as it is data processing related to the gait. For example, the result of the gait measurement by the data processing unit 127 is displayed on a display device (not illustrated). For example, the result of the gait measurement by the data processing unit 127 is output to a system using the result. A method of using the result of the gait measurement by the data processing unit 127 is not particularly limited.

### (Operation)

Next, an operation of the gait measurement system 1 will be described with reference to the drawings. Hereinafter, operations of the measurement device 11 and the data processing device 12 included in the gait measurement system 1 will be individually described.

### [Control Unit]

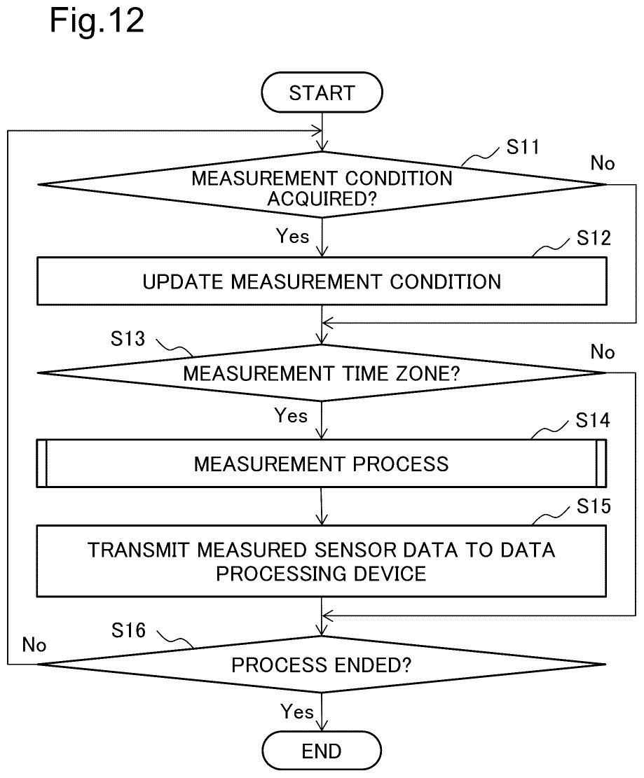

FIG. 12 is a flowchart for describing an example of the operation of the control unit 113 included in the measurement device 11. In the description along the flowchart of FIG. 12, the control unit 113 will be described as an operation subject.

In FIG. 12, first, when the measurement condition is acquired (Yes in step S11), the control unit 113 updates the measurement condition (step S12). In a case where the measurement condition has not been acquired (No in step S11), the process proceeds to step S13.

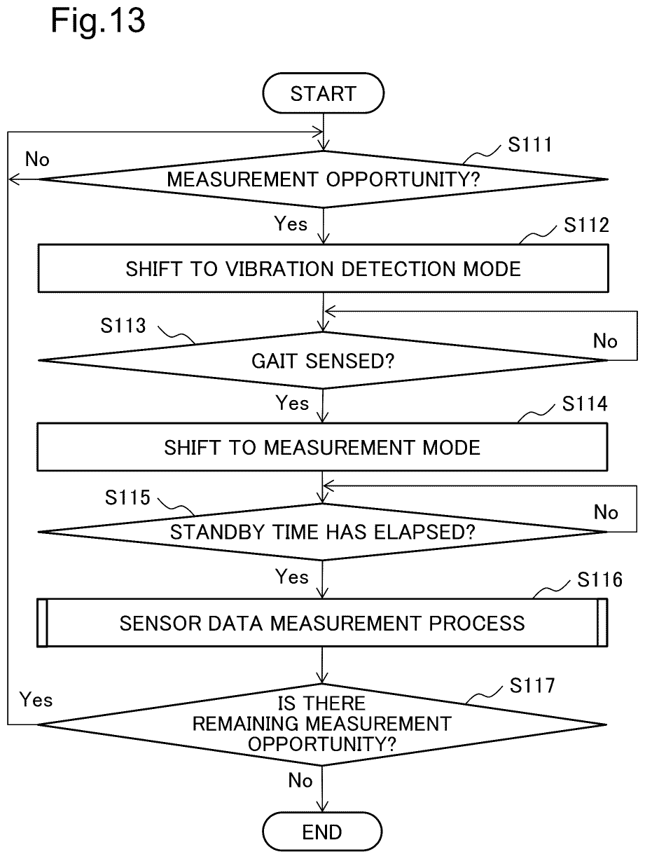

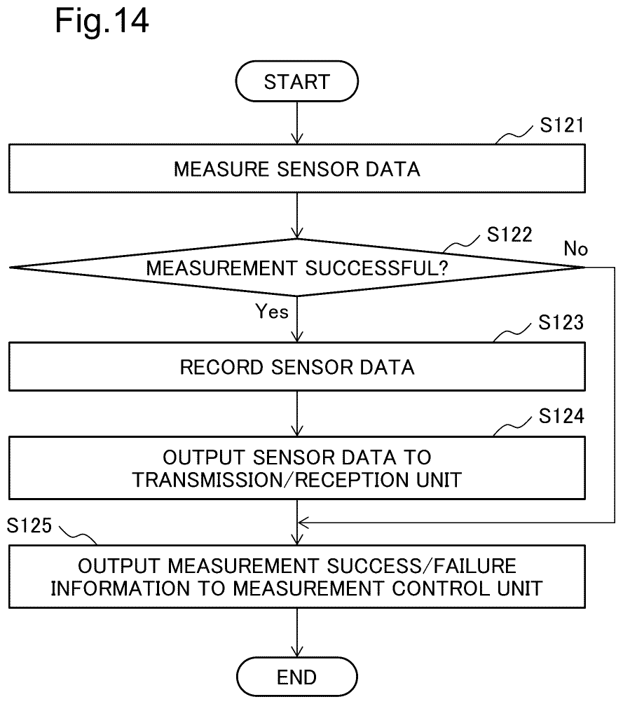

After step S12 or in the case of No in step S11, when the measurement time zone is reached (Yes in step S13), the control unit 113 executes the measurement process (step S14). Details of the measurement process will be described later (FIGS. 13 to 14). In a case where the measurement time zone is not reached (No in step S13), the process proceeds to step S16.

After step S14, the control unit 113 transmits the sensor data measured in the measurement process to the data processing device 12 (step S15).

After step S15 or in the case of No in step S13, when the process is continued (No in step S16), the process returns to step S11. In a case where the process is ended (Yes in step S16), the process along the flowchart in FIG. 12 is ended. The timing to end the process along the flowchart of FIG. 12 can be set to any timing. For example, the timing to end the process along the flowchart of FIG. 12 may be set in advance. For example, the process may be ended according to an instruction input by the user who uses the gait measurement system 1.

### [Measurement Process]

FIG. 13 is a flowchart for describing an example of a measurement process (step S14 in FIG. 12) of the control unit 113 included in the measurement device 11. In the description along the flowchart of FIG. 13, the control unit 113 will be described as an operation subject.

In FIG. 13, first, when a measurement opportunity arrives (Yes in step S111), the control unit 113 shifts to the vibration detection mode (step S112). When the mode shifts to the vibration detection mode, the acceleration sensor 111, the angular velocity sensor 112, and the control unit 113 are activated. In the vibration detection mode, the control unit 113 operates with low power saving and detects the gait according to values such as an acceleration and an angular velocity. In a case where the measurement opportunity has not arrived (No in step S111), the control unit 113 waits until the measurement opportunity arrives. For example, in preparation for an error in setting or counting a measurement opportunity, in a case where the measurement opportunity does not arrive in the measurement time zone, the measurement in the measurement time zone may be stopped (the process proceeds to step S16 in FIG. 12).

When the gait is sensed after shifting to the vibration detection mode in step S112 (Yes in step S113), the control unit 113 shifts to the measurement mode (step S114). In a case where the gait is not sensed (No in step S113), the control unit 113 waits until the gait is sensed. For example, there may be a situation where the gait is not detected after shifting to the measurement mode. In preparation for such a situation, the unit may be configured in such a way that the measurement at the measurement opportunity is forcibly started or the measurement at the measurement opportunity is stopped when a certain period of time elapses after the shift to the measurement mode.

After step S114, when the standby time has elapsed (Yes in step S115), the control unit 113 executes a sensor data measurement process (step S116). The sensor data measurement process in step S116 will be described later. In a case where the standby time has not elapsed (No in step S115), the control unit 113 waits until the standby time elapses.

After step S116, in a case where there is a remaining measurement opportunity (Yes in step S117), the process returns to step S111. On the other hand, in a case where there is no remaining measurement opportunity (No in step S117), the process along the flowchart of FIG. 13 ends (the process proceeds to step S16 of FIG. 12).

### [Sensor Data Measurement Process]

FIG. 14 is a flowchart for describing an example of a sensor data measurement process (step S116 in FIG. 13) of the control unit 113 included in the measurement device 11. In the description along the flowchart of FIG. 14, the control unit 113 will be described as an operation subject.

In FIG. 14, first, the control unit 113 measures sensor data in accordance with the timing at which the standby time has elapsed after shifting to the measurement mode (step S121). The control unit 113 measures sensor data based on sensor values measured by the acceleration sensor 111 and the angular velocity sensor.

When the measurement succeeds (Yes in step S122), the control unit 113 stores the measured sensor data in a storage unit (not illustrated) (step S123). In a case where the measurement fails (No in step S122), the process proceeds to step S125.

After step S123, the control unit 113 outputs the measured sensor data to the transmission/reception unit 116 (step S124). The sensor data output to the transmission/reception unit 116 is transmitted to the data processing device 12.

After step S124 or in the case of No in step S122, the control unit 113 outputs the measurement success/failure information to the condition setting unit 115 (step S125). The measurement success/failure information output to the condition setting unit 115 is used to set the measurement condition including the change of the standby time and the like.

### [Condition Setting Unit]

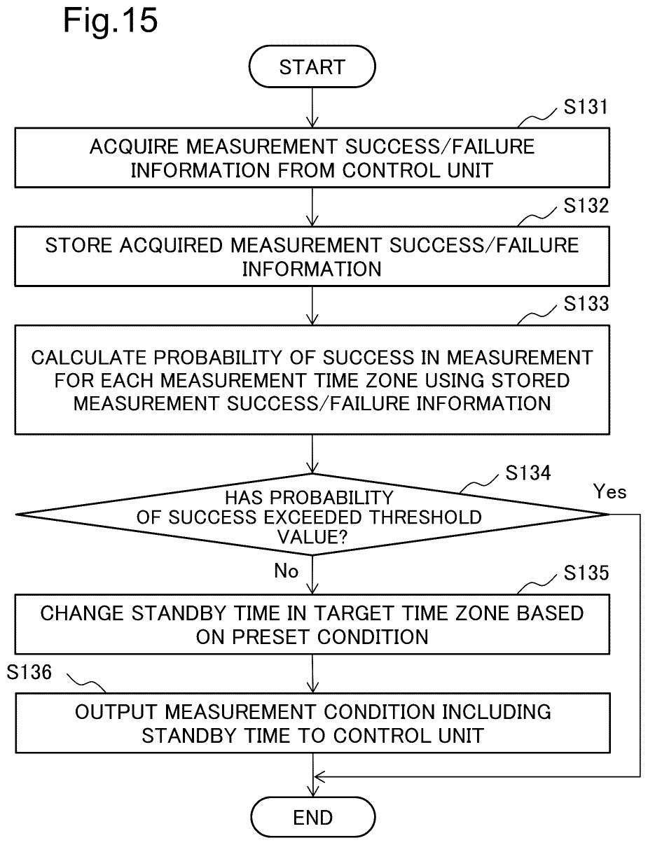

FIG. 15 is a flowchart for describing an example of the measurement condition generation process by the condition setting unit 115 included in the measurement device 11. In the description along the flowchart of FIG. 15, the condition setting unit 115 will be described as an operation subject.

In FIG. 15, first, the condition setting unit 115 acquires measurement success/failure information from the control unit 113 (step S131).

Next, the condition setting unit 115 stores the acquired measurement success/failure information (step S132).

Next, the condition setting unit 115 calculates a probability of success in measurement for each measurement time zone using the stored measurement success/failure information (step S133). In step S133, a probability of failure may be calculated instead of a probability of success.

In a case where the probability of success does not exceed the threshold value (No in step S134), the condition setting unit 115 changes the standby time in the target time zone based on a preset condition (step S135). In a case where the probability of success exceeds the threshold value (Yes in step S134), the process along the flowchart of FIG. 15 is ended.

After step S135, the condition setting unit 115 outputs the measurement condition including the standby time to the control unit 113 (step S136).

### [Data Processing Device]

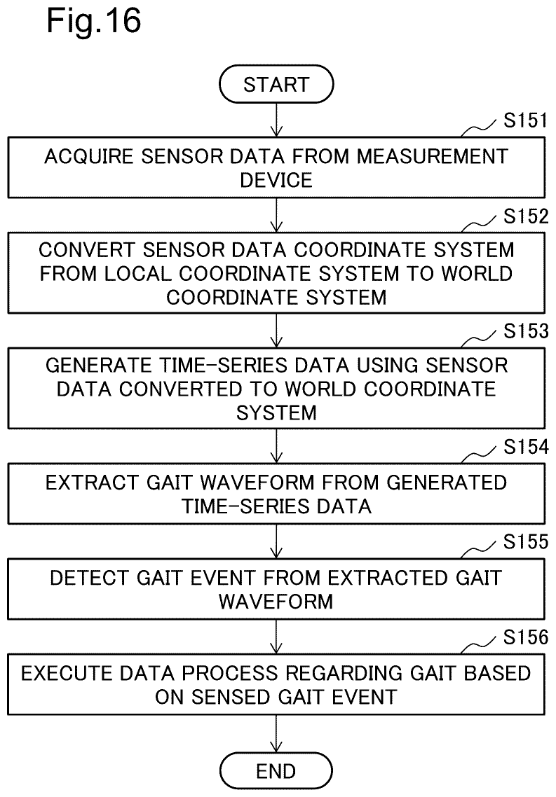

FIG. 16 is a flowchart for describing an example of the operation of the data processing device 12. In the description along the flowchart of FIG. 16, the data processing device 12 will be described as an operation subject.

In FIG. 16, first, the data processing device 12 acquires sensor data from the measurement device 11 (step S151).

Next, the data processing device 12 converts the coordinate system of the sensor data from the local coordinate system to the world coordinate system (step S152).

Next, the data processing device 12 generates time-series data using the sensor data converted into the world coordinate system (step S153). For example, the data processing device 12 generates time-series data of an acceleration, a velocity, and a position (trajectory) in each of the three axis directions, and time-series data of an angular velocity and an angle around each of the three axes.

Next, the data processing device 12 extracts a gait waveform from the generated time-series data (step S154).

Next, the data processing device 12 detects a gait event from the extracted gait waveform (step S155).

Next, the data processing device 12 executes a data process regarding the gait based on the sensed gait event (step S156). For example, the data processing device 12 estimates the physical characteristics and physical condition of the user based on the sensed gait event.

As described above, the gait measurement system of the present example embodiment includes the measurement device and the data processing device. The gait measurement system of the present example embodiment is characterized in that the condition setting device is included in the measurement device.

The measurement device includes a condition setting unit (condition setting device), a sensor, a control unit, and a data transmission/reception unit. The condition setting unit acquires measurement success/failure information indicating success/failure of the measurement of the physical quantity related to the motion of the foot executed by the measurement device installed at the foot portion of the user at the measurement opportunity included in the target measurement time zone. The condition setting unit sets a measurement condition including a standby time from when the gait is sensed at a measurement opportunity included in a related measurement time zone related to the target measurement time zone to when the measurement is started based on the measurement success/failure information in the target measurement time zone. The sensor measures the spatial acceleration and the spatial angular velocity. The control unit is activated at at least one measurement opportunity included in the measurement time zone based on the measurement condition set by the condition setting device. The control unit generates sensor data based on the spatial acceleration and the spatial angular velocity measured by the sensor. The data transmission/reception unit transmits the sensor data to the data processing device. The data transmission/reception unit receives the input information input by the user from the data processing device.

The data processing device includes an operation reception unit, a transmission/reception unit, a generation unit, a sensing unit, and a data processing unit. The operation reception unit receives input information input according to a user's operation. The transmission/reception unit receives sensor data related to the motion of the foot measured by a measurement device installed at the foot portion of the user. The generation unit generates a gait waveform that is time-series data of the sensor data received by the transmission/reception unit. The sensing unit detects a gait event from the gait waveform generated by the generation unit. The data processing unit executes a data process using the gait event sensed by the sensing unit.

In the present example embodiment, the measurement device varies and sets the measurement condition including the standby time from the detection of the gait of the user to the start of the measurement in accordance with the lifestyle of the user. Therefore, according to the present example embodiment, by setting an appropriate standby time according to the user's lifestyle, it is possible to set the measurement condition for performing the gait measurement at an appropriate timing while reducing the power consumption. When the power consumption of the measurement device is reduced, the battery life is extended. Instead of extending the battery life, the power consumption to be reduced may be allocated to another data processing to enhance the service content.

In general gait measurement, since gait measurement is performed at an any timing when the stable gait is sensed, consumption of a battery built in a measurement device is severe. The measurement device can be used for a long period of time in a use environment in which frequent battery replacement and charging are possible. However, in order to improve usability, frequent battery replacement and charging are not preferable. Therefore, it is required to enable stable gait measurement for a long period of time without requiring battery replacement. In order to stably perform the gait measurement in the long term without replacing the battery, the measurement time zone of the gait may be limited in accordance with the lifestyle of the user. However, even when the measurement time zone of the gait is matched to the lifestyle of the user, the gait of the user in each measurement time zone is not always constant, and thus the gait measurement may fail. For example, immediately after the user starts a stable gait and starts measurement, a change in the gait state occurs in scenes such as ascent/descent of stairs, use of an elevator, selection and accounting of products in a store such as a supermarket, and pick-up and drop-off of a child. When the gait state changes every time the measurement opportunity included in the measurement time zone occurs, measurement failure frequently occurs. According to the method of the present example embodiment, in the measurement time zone set in accordance with the lifestyle of the user, the standby time from the detection of the stable gait to the start of the measurement can be set according to the gait characteristic of the user, and thus the measurement failure hardly occurs.

In an aspect of the present example embodiment, the acquisition unit acquires the measurement success/failure information including a time and a number of times of occurrence of the measurement failure in the measurement opportunity included in the target measurement time zone. The measurement condition setting unit changes the standby time at the measurement opportunity included in the related measurement time zone related to the target measurement time zone based on the time and a number of times of occurrence of the measurement failure in the measurement opportunity included in the target measurement time zone. In the present aspect, the standby time of the measurement opportunity included in the related measurement time zone is changed based on the time and a number of times of occurrence of the measurement failures in the measurement time zone of the target measurement time zone. Therefore, according to the present aspect, it is possible to set a measurement condition in which the failure is unlikely to occur in measurement in a measurement time zone in which a measurement failure is likely to occur.

In an aspect of the present example embodiment, the measurement condition setting unit sets the standby time at the measurement opportunity included in the related measurement time zone of the same time zone subsequent to the target measurement time zone based on the measurement success/failure information in the target measurement time zone. The user tends to act in a similar pattern in the same time zone in his or her lifestyle. In the present aspect, by setting the standby time at the measurement opportunity included in the related measurement time zone of the same time zone subsequent to the target measurement time zone, the standby time is set in accordance with the lifestyle of the user. Therefore, according to the present aspect, it is possible to perform gait measurement customized for the life of the user by setting the standby time in accordance with the lifestyle of the user.

A condition setting device according to an aspect of the present example embodiment includes a calculation unit that calculates a probability of success in measurement in at least one measurement opportunity included in a target measurement time zone based on measurement success/failure information in the target measurement time zone. The measurement condition setting unit sets the standby time in which the probability of success exceeds the threshold value as the standby time at the measurement opportunity included in the related measurement time zone related to the target measurement time zone. According to the present aspect, the accuracy of the gait measurement is improved by setting the standby time according to the probability of success in measurement.

A condition setting device according to an aspect of the present example embodiment includes a calculation unit that calculates a probability of success in measurement in at least one measurement opportunity included in a target measurement time zone based on measurement success/failure information in the target measurement time zone. Based on the measurement success/failure information in the target measurement time zone, the measurement condition setting unit sets the standby times at the plurality of measurement opportunities included in the related measurement time zone related to the target measurement time zone to different times. The measurement condition setting unit sets the standby time in which the probability of success in measurement is maximum in at least one measurement opportunity included in the related measurement time zone as the standby time at the measurement opportunity included in the measurement time zone related to the related measurement time zone. According to the present aspect, the accuracy of the gait measurement is further improved by setting the standby time in which the probability of success in measurement is maximum.

### Second Example Embodiment

Next, a gait measurement system according to a second example embodiment will be described with reference to the drawings. A gait measurement system of the present example embodiment is different from that of the first example embodiment in that instead of a measurement device, a data measurement device includes a condition setting unit.

### (Configuration)

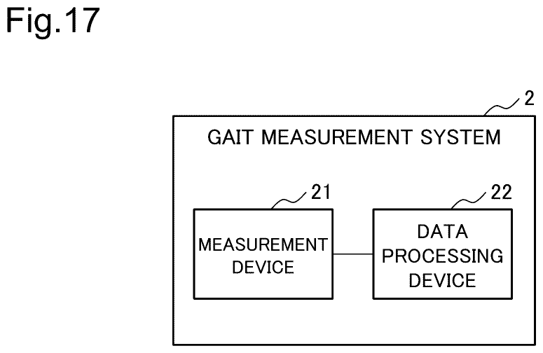

FIG. 17 is a block diagram illustrating a configuration of a gait measurement system 2 of the present example embodiment. The gait measurement system 2 includes a measurement device 21 and a data processing device 22. The measurement device 21 and the data processing device 22 may be connected by wire or wirelessly. The measurement device 21 and the data processing device 22 may be configured by a single device. Hereinafter, the measurement device 21 and the data processing device 22 will be individually described.

### [Measurement Device]

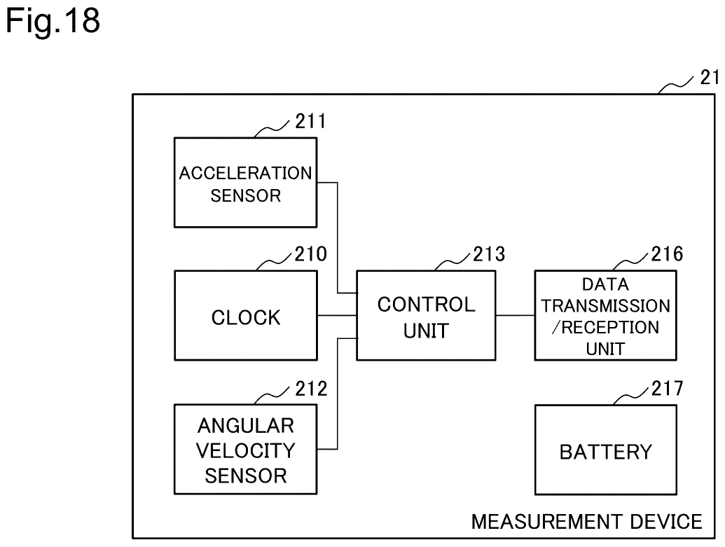

FIG. 18 is a block diagram illustrating an example of a detailed configuration of the measurement device 21. The measurement device 21 includes a clock 210, an acceleration sensor 211, an angular velocity sensor 212, a control unit 213, a transmission/reception unit 216, and a battery 217. The measurement device 21 has a configuration in which the measurement condition setting unit 156 is removed from the measurement device 11 of the first example embodiment.

The clock 210 is a real-time clock that ticks time. The clock 210 has a configuration similar to that of the clock 110 of the first example embodiment.

The acceleration sensor 211 is a sensor that measures acceleration (also referred to as spatial accelerations) in the three axial directions. The acceleration sensor 211 has a configuration similar to that of the acceleration sensor 111 of the first example embodiment. The acceleration sensor 211 measures accelerations in the three axial directions according to the set measurement conditions. The acceleration sensor 211 outputs the measured acceleration to the control unit 213.

The angular velocity sensor 212 is a sensor that measures angular velocities in the three axial directions (also referred to as spatial angular velocities). The angular velocity sensor 212 has a configuration similar to that of the angular velocity sensor 112 of the first example embodiment. The angular velocity sensor 212 measures angular velocities in the three axial directions according to the set measurement condition. Angular velocity sensor 212 outputs the measured angular velocity to control unit 213.

The control unit 213 has a configuration similar to that of the control unit 113 of the first example embodiment. The control unit 213 acquires the acceleration in each of the three-axis directions and the angular velocity around each of the three axes from the acceleration sensor 211 and the angular velocity sensor 212, respectively, according to the set measurement condition. The control unit 213 converts the acquired acceleration and angular velocity into digital data to output the converted digital data (also referred to as sensor data) to the transmission/reception unit 216. The sensor data includes measurement times of the acceleration and the angular velocity. The control unit 213 outputs measurement success/failure information of the measured sensor data to the transmission/reception unit 216.

The transmission/reception unit 216 has a configuration similar to that of the transmission/reception unit 116 of the first example embodiment. The transmission/reception unit 216 acquires sensor data from the control unit 213. The transmission/reception unit 216 acquires the measurement success/failure information of the sensor data from the control unit 213. In a case where the measurement is successful, the transmission/reception unit 216 transmits the sensor data and the measurement success/failure information to the data processing device 22. In a case where the measurement is successful, the transmission/reception unit 216 may transmit only the sensor data to the data processing device 22. In a case where the measurement fails, the transmission/reception unit 216 transmits the measurement success/failure information to the data processing device 22. Even in a case where the measurement fails, when the sensor data is measured, the transmission/reception unit 216 may transmit the sensor data to the data processing device 22.

The transmission/reception unit 216 receives the measurement condition from the data processing device 22. The measurement condition received by the transmission/reception unit 216 is set as a condition for measurement by the measurement device 21. The measurement condition includes a standby time in the measurement time zone. The set standby time may be reflected in a measurement time zone immediately after reception or may be reflected in a measurement time zone on or after the next day. In order to set the standby time according to the user's habit, it is preferable that the standby time is reflected in the same measurement time zone on or after the next day.

The battery 217 is a power source for components included in the measurement device 21. The battery 217 has a configuration similar to that of the battery 117 of the first example embodiment. The power of the battery 217 is consumed in the operations of the clock 210, the acceleration sensor 211, the angular velocity sensor 212, the control unit 213, and the transmission/reception unit 216.

### [Data Processing Device]

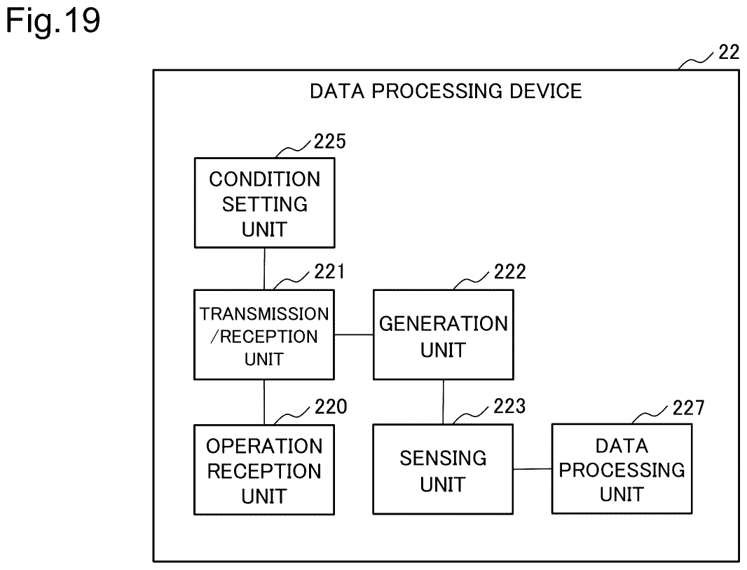

FIG. 19 is a block diagram illustrating an example of a configuration of the data processing device 22. The data processing device 22 includes an operation reception unit 220, a transmission/reception unit 221, a generation unit 222, a sensing unit 223, a condition setting unit 225, and a data processing unit 227. The data processing device 22 has a configuration in which the condition setting unit 115 of the first example embodiment is added to the data processing device 12 of the first example embodiment. The condition setting unit 225 may be configured as a single device (condition setting device).

The operation reception unit 220 receives information (also referred to as input information) input by the user. The operation reception unit 220 has a configuration similar to that of the operation reception unit 120 of the first example embodiment. For example, the operation reception unit 220 may receive the standby time set by the user as the input information. The operation reception unit 220 outputs input information to the transmission/reception unit 221.

The transmission/reception unit 221 receives the sensor data and the measurement success/failure information from the measurement device 21. The transmission/reception unit 221 has a configuration similar to that of the transmission/reception unit 121 of the first example embodiment. The transmission/reception unit 221 outputs the received sensor data to the generation unit 222. The transmission/reception unit 221 outputs the received measurement success/failure information to the condition setting unit 225. The transmission/reception unit 221 acquires the measurement condition from the condition setting unit 225. The transmission/reception unit 221 transmits the acquired measurement conditions to the measurement device 21. Further, the transmission/reception unit 221 acquires input information related to an operation by the user from the operation reception unit 220. The transmission/reception unit 221 transmits the input information to the condition setting unit 225 and the measurement device 21.

The generation unit 222 has a configuration similar to that of the generation unit 122 of the first example embodiment. The generation unit 222 acquires sensor data from the transmission/reception unit 221. The generation unit 222 converts the coordinate system of the acquired sensor data from the local coordinate system to the world coordinate system. The generation unit 222 generates time-series data (also referred to as a gait waveform) of the sensor data after conversion into the world coordinate system. The generation unit 222 outputs the generated gait waveform to the sensing unit 223.

The sensing unit 223 has a configuration similar to that of the sensing unit 123 of the first example embodiment. The sensing unit 223 acquires the gait waveform from the generation unit 222. The sensing unit 223 detects a gait event from the gait waveform. The sensing unit 223 outputs, to the data processing unit 227, data used for gait measurement such as the timing of the sensed gait event and a value of sensor data in a predetermined period with the timing of the gait event as the starting point.

The condition setting unit 225 acquires the measurement success/failure information in the measurement time zone from the transmission/reception unit 221. The condition setting unit 225 has a configuration similar to that of the condition setting unit 115 of the first example embodiment. The condition setting unit 225 records the acquired measurement success/failure information. The measurement success/failure information includes information such as a measurement time, an activation condition, and trial count information indicating at which measurement opportunity the measurement was successful. The measurement success/failure information may include sensor data. The measurement success/failure information may include a success flag in a case where the measurement succeeds in the measurement time zone and a failure flag in a case where the measurement fails in the measurement time zone. The measurement success/failure information to which the success flag is attached is also referred to as success information, and the measurement success/failure information to which the failure flag is attached is also referred to as failure information. The condition setting unit 225 registers, in a storage unit (not illustrated), failure information including a date and time of a measurement time zone in a case where measurement has been executed but the measurement has not been completed and the number times of the failure in the measurement time zone.

The condition setting unit 225 sets the standby time in the measurement time zone subsequent to the measurement time zone according to the measurement success/failure information for each measurement time zone. For example, the condition setting unit 225 changes the standby time in the target time zone based on failure information in the target time zone (also referred to as target time zone) in which the standby time is to be changed. For example, the condition setting unit 225 changes the standby time in the target time zone according to the probability of success or the probability of failure in measurement in the target time zone in which the standby time has been changed.

For example, the condition setting unit 225 sets the standby time based on failure factors such as the number of times of measurement failure and the timing when the failure occurs. For example, the condition setting unit 225 changes the standby time in the target time zone a plurality of times, and selects the standby time in which a probability of success in measurement is maximum. For example, the condition setting unit 225 repeats updating of the standby time until the probability of success in measurement exceeds the threshold value. For example, the condition setting unit 225 may randomly set the standby time in each of a plurality of measurement opportunities included in the same measurement time zone, and set the standby time having a high probability of success.

For example, the condition setting unit 225 may set a standby time with a high probability of success in measurement in a certain measurement time zone as a standby time in another measurement time zone. For example, the condition setting unit 225 may determine a factor of measurement failure by weighting with a month, a week, a time, the number of times, or the like, and set the standby time according to the month, the week, the time, the number of times. For example, the condition setting unit 225 may record the optimal standby time for each day of the week or time and set the standby time according to the day of the week or time. For example, the condition setting unit 225 may reset the standby time at the timing of the change of seasons. For example, in a case where the data processing device 22 is achieved by an application installed in a mobile terminal, the condition setting unit 225 may set the standby time using position information acquired by a global positioning system (GPS) of the mobile terminal. For example, when the gait position of the user can be identified by the position information, it can be determined whether there is a high possibility that a stable gait is performed.

In a case where the measurement device 21 is mounted on the footwear of both right and left feet, there is a possibility that the measurement time zones and the standby times set for the right and left feet are greatly deviated due to individual differences of the measurement device 21, the dominant foot of the user, and the like. For example, for a user who steps out from the left foot when the user starts the gait from a stopped state, the timing of shifting to a stable gait is earlier in the left foot. For example, in a case where there are many curves on the road on which the user walks, when the standby time is set in accordance with the timing of the gait on the curve, the deviation between the left and right standby times may be large. When the deviation between the left and right standby times increases, the standby time for one foot is longer for the gait in a normal time, and the effect of reducing power consumption may be reduced. Therefore, it is preferable that the standby time does not greatly deviate between the left and right legs. For example, in a case where the standby times of the measurement devices 21 mounted on the footwear of both the right and left feet deviates beyond the identified value, an algorithm for reducing the deviation between the standby times of the measurement device 21 may be added to the setting of the measurement condition by the condition setting unit 225.

The data processing unit 227 has a configuration similar to that of the data processing unit 227 of the first example embodiment. The data processing unit 227 acquires data used for gait measurement from the sensing unit 223. The data processing unit 227 performs gait measurement using the acquired data. For example, the result of the gait measurement by the data processing unit 227 is displayed on a display device (not illustrated). For example, the result of the gait measurement by the data processing unit 227 is output to a system using the result. A method of using the result of the gait measurement by the data processing unit 227 is not particularly limited.

As described above, the gait measurement system of the present example embodiment includes the measurement device and the data processing device. The gait measurement system of the present example embodiment is characterized in that the condition setting device is included in the data processing device.

The measurement device includes a sensor, a control unit, and a data transmission/reception unit. The sensor measures the spatial acceleration and the spatial angular velocity. The control unit is activated at at least one measurement opportunity included in the measurement time zone based on the measurement condition set by the condition setting device. The control unit generates sensor data based on the spatial acceleration and the spatial angular velocity measured by the sensor. The data transmission/reception unit transmits the sensor data to the data processing device. The data transmission/reception unit receives input information input by the user and measurement conditions set by the data processing device from the data processing device.

The data processing device includes a condition setting unit (condition setting device), an operation reception unit, a transmission/reception unit, a generation unit, a sensing unit, and a data processing unit. The condition setting unit acquires measurement success/failure information indicating success/failure of the measurement of the physical quantity related to the motion of the foot executed by the measurement device installed at the foot portion of the user at the measurement opportunity included in the target measurement time zone. The condition setting unit sets a measurement condition including a standby time from when the gait is sensed at a measurement opportunity included in a related measurement time zone related to the target measurement time zone to when the measurement is started based on the measurement success/failure information in the target measurement time zone. The operation reception unit receives input information input according to a user's operation. The transmission/reception unit receives sensor data related to the motion of the foot measured by a measurement device installed at the foot portion of the user. The transmission/reception unit transmits the input information input by the user and the measurement condition set by the condition setting device to the measurement device. The generation unit generates a gait waveform that is time-series data of the sensor data received by the transmission/reception unit. The sensing unit detects a gait event from the gait waveform generated by the generation unit. The data processing unit executes a data process using the gait event sensed by the sensing unit.

In the present example embodiment, the data processing device varies and sets the measurement condition including the standby time from the detection of the gait of the user to the start of the measurement in accordance with the lifestyle of the user. Therefore, according to the present example embodiment, by setting an appropriate standby time according to the user's lifestyle, it is possible to set the measurement condition for performing the gait measurement at an appropriate timing while reducing the power consumption. According to the present example embodiment, the measurement device sets the measurement condition, whereby the power consumption of the measurement device is reduced as compared with that in the first example embodiment.

In an aspect of the present example embodiment, the condition setting device changes the standby time of at least one of the measurement devices installed at the left and right feet of the user in a case where the standby times included in the measurement conditions of the measurement devices installed at the left and right feet of the user deviate beyond a prescribed value. The condition setting device reduces a deviation between the standby times of the measurement devices installed at the left and right feet of the user. According to the present aspect, it is possible to further improve the accuracy of the gait measurement by alleviating the deviation between the standby times included in the measurement conditions of the measurement devices installed at the left and right feet.

### Third Example Embodiment

Next, a condition setting device according to the third example embodiment will be described with reference to the drawings. The gait measurement system of the present example embodiment has a configuration in which the condition setting unit (condition setting device) of each example embodiment is simplified.

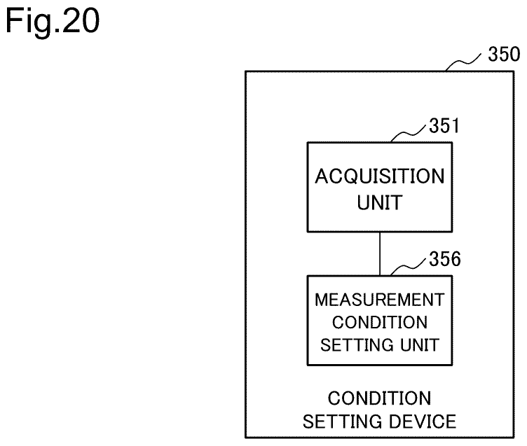

FIG. 20 is a block diagram illustrating an example of a configuration of a condition setting device 350 according to the present example embodiment. The condition setting device 350 includes an acquisition unit 351 and a measurement condition setting unit 356. The acquisition unit 351 acquires measurement success/failure information indicating success/failure of the measurement of the physical quantity related to the motion of the foot executed by the measurement device installed at the foot portion of the user at the measurement opportunity included in the target measurement time zone. The measurement condition setting unit 356 sets a measurement condition including a standby time from when the gait is sensed at a measurement opportunity included in a related measurement time zone related to the target measurement time zone to when the measurement is started based on the measurement success/failure information in the target measurement time zone.

In the present example embodiment, the measurement condition including the standby time from the detection of the gait of the user to the start of the measurement is varied and set in accordance with the lifestyle of the user. Therefore, according to the present example embodiment, by setting an appropriate standby time according to the user's lifestyle, it is possible to set the measurement condition for performing the gait measurement at an appropriate timing while reducing the power consumption.

### (Hardware)

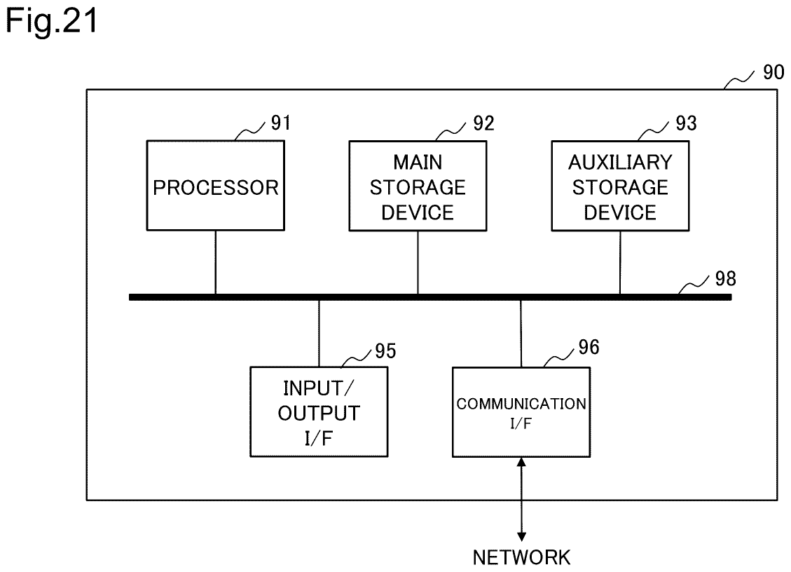

A hardware configuration for executing control and processing according to each example embodiment of the present disclosure will be described using an information processing device 90 of FIG. 21 as an example. The information processing device 90 in FIG. 21 is a configuration example for performing control and a process of each example embodiment, and does not limit the scope of the present disclosure.

As illustrated in FIG. 21, the information processing device 90 includes a processor 91, a main storage device 92, an auxiliary storage device 93, an input/output interface 95, and a communication interface 96. In FIG. 21 the interface is abbreviated as an interface (I/F). The processor 91, the main storage device 92, the auxiliary storage device 93, the input/output interface 95, and the communication interface 96 are data-communicably connected to each other via a bus 98. The processor 91, the main storage device 92, the auxiliary storage device 93, and the input/output interface 95 are connected to a network such as the Internet or an intranet via the communication interface 96.

The processor 91 develops the program stored in the auxiliary storage device 93 or the like in the main storage device 92. The processor 91 executes the program developed in the main storage device 92. In the present example embodiment, a software program installed in the information processing device 90 may be used. The processor 91 executes control and processing according to the present example embodiment.

The main storage device 92 has an area in which a program is developed. A program stored in the auxiliary storage device 93 or the like is developed in the main storage device 92 by the processor 91. The main storage device 92 is achieved by, for example, a volatile memory such as a dynamic random access memory (DRAM). A nonvolatile memory such as a magnetoresistive random access memory (MRAM) may be configured and added as the main storage device 92.

The auxiliary storage device 93 stores various pieces of data such as programs. The auxiliary storage device 93 is achieved by a local disk such as a hard disk or a flash memory. Various pieces of data may be stored in the main storage device 92, and the auxiliary storage device 93 may be omitted.

The input/output interface 95 is an interface that connects the information processing device 90 with a peripheral device based on a standard or a specification. The communication interface 96 is an interface that connects to an external system or a device through a network such as the Internet or an intranet in accordance with a standard or a specification. The input/output interface 95 and the communication interface 96 may be shared as an interface connected to an external device.

An input device such as a keyboard, a mouse, or a touch panel may be connected to the information processing device 90 as necessary. These input devices are used to input of information and settings. In a case where the touch panel is used as the input device, the display screen of the display device may also serve as the interface of the input device. Data communication between the processor 91 and the input device may be mediated by the input/output interface 95.

The information processing device 90 may be provided with a display device that displays information. In a case where a display device is provided, the information processing device 90 preferably includes a display control device (not illustrated) that controls display of the display device. The display device may be connected to the information processing device 90 via the input/output interface 95.

The information processing device 90 may be provided with a drive device. The drive device mediates reading of data and a program from the recording medium, writing of a processing result of the information processing device 90 to the recording medium, and the like between the processor 91 and the recording medium (program recording medium). The drive device may be connected to the information processing device 90 via the input/output interface 95.

The above is an example of a hardware configuration for enabling control and processing according to each example embodiment of the present invention. The hardware configuration of FIG. 21 is an example of a hardware configuration for executing control and processing according to each example embodiment, and does not limit the scope of the present invention. A program for causing a computer to execute processing related to control and a process according to each example embodiment is also included in the scope of the present invention. A program recording medium in which the program according to each example embodiment is recorded is also included in the scope of the present invention. The recording medium can be achieved by, for example, an optical recording medium such as a compact disc (CD) or a digital versatile disc (DVD). The recording medium may be achieved by a semiconductor recording medium such as a Universal Serial Bus (USB) memory or a secure digital (SD) card. The recording medium may be achieved by a magnetic recording medium such as a flexible disk, or another recording medium. In a case where the program executed by the processor is recorded in the recording medium, the recording medium is a program recording medium.

The components of each example embodiment may be combined in any manner. The components of each example embodiment may be achieved by software or may be achieved by a circuit.

While the present invention is described with reference to example embodiments thereof, the present invention is not limited to these example embodiments. Various modifications that can be understood by those of ordinary skill in the art can be made to the configuration and details of the present invention within the scope of the present invention.

### REFERENCE SIGNS LIST

1, 2 gait measurement system
11, 21 measurement device
12, 22 data processing device
110, 210 clock
111, 211 acceleration sensor
112, 212 angular velocity sensor
113, 213 control unit
115, 225 condition setting unit
116, 216 transmission/reception unit
117, 217 battery
120, 220 operation reception unit
121, 221 transmission/reception unit
122, 222 generation unit
123, 223 sensing unit
127, 227 data processing unit
151 acquisition unit
153 storage unit
155 calculation unit
156 measurement condition setting unit
157 output unit
350 condition setting device
351 acquisition unit
356 measurement condition setting unit

What is claimed is:

1. A condition setting device comprising:
a first memory storing instructions; and
a first processor connected to the first memory and configured to execute the instructions to:
acquire measurement success/failure information indicating success/failure of measurement of a physical quantity related to a motion of a foot, the measurement being executed by a measurement device installed at a foot portion of a user at a measurement opportunity included in a target measurement time zone; and
set, based on the measurement success/failure information in the target measurement time zone, a measurement condition including a standby time from when a gait is sensed at a measurement opportunity included in a related measurement time zone related to the target measurement time zone to when measurement is started.

2. The condition setting device according to claim 1, wherein
the first processor is configured to execute the instructions to
acquire the measurement success/failure information including a time and a number of times of occurrence of a measurement failure at the measurement opportunity included in the target measurement time zone, and
change the standby time at the measurement opportunity included in the related measurement time zone related to the target measurement time zone based on a time and a number of times of occurrence of the measurement failure at the measurement opportunity included in the target measurement time zone.

3. The condition setting device according to claim 1, wherein
the first processor is configured to execute the instructions to
set the standby time at a measurement opportunity included in the related measurement time zone of the same time zone subsequent to the target measurement time zone based on the measurement success/failure information in the target measurement time zone.

4. The condition setting device according to claim 1, wherein
the first processor is configured to execute the instructions to
calculate a probability of success in measurement at at least one measurement opportunity included in the target measurement time zone based on the measurement success/failure information in the target measurement time zone, and
set the standby time in which the probability of success exceeds a threshold value as the standby time at a measurement opportunity included in the related measurement time zone related to the target measurement time zone.

5. The condition setting device according to claim 1, wherein
the first processor is configured to execute the instructions to
calculate a probability of success in measurement at at least one measurement opportunity included in the target measurement time zone based on the measurement success/failure information in the target measurement time zone,
set standby times at a plurality of measurement opportunities included in the related measurement time zone related to the target measurement time zone to different times based on the measurement success/failure information in the target measurement time zone, and
set the standby time at which the probability of success in measurement at at least one measurement opportunity included in the related measurement time zone is maximum as the standby time at a measurement opportunity included in the measurement time zone related to the related measurement time zone.

6. A measurement device comprising:
the condition setting device according to claim 1;
a sensor that measures a spatial acceleration and a spatial angular velocity; and
a controller configured to generate sensor data based on the spatial acceleration and the spatial angular velocity measured by the sensor, the controller being activated at at least one measurement opportunity included in a measurement time zone based on a measurement condition set by the condition setting device.

7. A data processing device comprising:
the condition setting device according to claim 1;
a second memory storing instructions; and
a second processor connected to the second memory and configured to execute the instructions to:
receive input information input according to an operation of a user;
receive sensor data regarding a motion of a foot measured by a measurement device installed at a foot portion of the user;
generate a gait waveform that is time-series data of the sensor data;
sense a gait event from the gait waveform;
execute a data process using the gait event, and
transmit the input information and a measurement condition set by the condition setting device to the measurement device installed at the foot portion of the user.

8. The data processing device according to claim 7, wherein
in a case where standby times included in the measurement conditions of the measurement devices installed at left and right feet of the user deviate beyond a prescribed value, the second processor is configured to execute the instructions to
change a standby time of at least one of the measurement devices installed at the left and right feet of the user, and reduces a deviation between the standby times of the measurement devices installed at the left and right feet of the user.

9. A condition setting method executed by a computer, the method comprising:
acquiring measurement success/failure information indicating success/failure of measurement of a physical quantity related to a motion of a foot, the measurement being executed by a measurement device installed at a foot portion of a user at a measurement opportunity included in a target measurement time zone; and
setting, based on the measurement success/failure information in the target measurement time zone, a measurement condition including a standby time from when a gait is sensed at a measurement opportunity included in a related measurement time zone related to the target measurement time zone to when measurement is started.

10. A non-transitory recording medium storing a program for causing a computer to execute:
a process of acquiring measurement success/failure information indicating success/failure of measurement of a physical quantity related to a motion of a foot, the measurement being executed by a measurement device installed at a foot portion of a user at a measurement opportunity included in a target measurement time zone; and
a process of setting, based on the measurement success/failure information in the target measurement time zone, a measurement condition including a standby time from when a gait is sensed at a measurement opportunity included in a related measurement time zone related to the target measurement time zone to when measurement is started.
